(19) United States
(12) Patent Application Publication
(10) Pub. No.: US 2025/0329421 A1
(43) Pub. Date: Oct. 23, 2025

(54) INFORMATION PROCESSING APPARATUS, INFORMATION PROCESSING METHOD, AND COMPUTER-READABLE MEDIUM

(71) Applicant: Preferred Networks, Inc., Tokyo (JP)

(72) Inventor: Hodaka MORI, Tokyo (JP)

(73) Assignee: Preferred Networks, Inc., Tokyo (JP)

(21) Appl. No.: 19/182,118

(22) Filed: Apr. 17, 2025

(30) Foreign Application Priority Data
Apr. 22, 2024 (JP) 2024-069316

(51) Int. Cl. G16C 20/10; G16C 20/20; G16C 20/70

(52) U.S. Cl. CPC G16C 20/10; G16C 20/20; G16C 20/70

(57) ABSTRACT

An information processing apparatus according to an embodiment includes at least one memory, and at least one processor. The at least one processor is configured to: identify target atoms subject to chemical bonding among atoms; acquire information regarding a first action force acting on each of the atoms, the information being generated by inputting an atomic structure of the atoms into a neural network; acquire information regarding a first additional force to be applied to at least one of the target atoms; and execute a molecular dynamics simulation for the atoms using the information regarding the first action force, the information regarding the first additional force, and position information of the atoms.

### CROSS-REFERENCE TO RELATED APPLICATIONS

This application is based upon and claims the benefit of priority from Japanese Patent Application No. 2024-069316, filed on Apr. 22, 2024; the entire contents of all of which are incorporated herein by reference.

### BACKGROUND OF THE INVENTION

### 1. Field of the Invention

Embodiments described herein relate generally to information processing apparatuses, information processing methods, and a computer-readable medium.

### 2. Description of the Related Art

Conventionally, neural networks that predict the total energy of the atomic system and the force acting on individual atoms (hereafter referred to as neural network potentials (NNPs)) have been developed. Compared to electronic state simulations such as density functional theory (DFT), NNPs are capable of outputting energy and/or forces in a significantly shorter time. NNPs enable highly accurate and generalizable calculations for various substances (spanning a broad range of elements and structures). For example, NNPs are capable of handling chemical reactions due to their ability to accommodate diverse elements.

Additionally, ReaxFF is a similar technology to NNP. ReaxFF requires the determination of parameters for each type of chemical reaction, which has limitations in simulating a broad range of substances.

On the other hand, even if a molecular dynamics (MD) simulation is performed using NNP, chemical reactions take timescales and are not ordinarily observed. Thus, dynamically simulating reaction processes within a practical computational timeframe is challenging. For these reasons, it is difficult to handle chemical reactions such as polymerization and decomposition directly with the MD simulations using NNPs. Furthermore, simulating a chemical reaction using DFT is highly time-consuming, making it difficult to dynamically simulate reaction processes within a practical computational timeframe.

The related techniques are described in Behrouz Arash, Barend J. Thijsse, Alessandro Pecenko, Angelo Simone, “Effect of water content on the thermal degradation of amorphous polyamide 6, 6: A collective variable-driven hyperdynamics study” Polymer Degradation and Stability, Volume 146, in 2017, on pages 260-266, and Aniruddh Vashisth, Chowdhury Ashraf, Weiwei Zhang, Charles E Bakis, Adri C. T. van Duin, “Accelerated ReaxFF simulations for Describing the Reactive Cross-Linking of Polymers” The Journal of Physical Chemistry A 2018 Jul. 11, Volume 122, No. 32, on pages 6633-6642, American Chemical Society, URL:https://scholar.google.co.jp/citations?view_op=view_citation&hl=ja&user=XAMQip0AAAAJ&citation_for_view=XAMQip0AAAA J:4TOpqqG69KYC

The present disclosure is intended to solve the challenge of enabling the occurrence of a chemical reaction within a practical computational timeframe in a molecular dynamics simulation of the chemical reaction.

### SUMMARY OF THE INVENTION

An information processing apparatus according to an embodiment includes at least one memory, and at least one processor. The at least one processor is configured to: identify a plurality of target atoms subject to chemical bonding among a plurality of atoms; acquire information regarding a first action force acting on each of the plurality of atoms, the information being generated by inputting an atomic structure of the plurality of atoms into a neural network; acquire information regarding a first additional force to be applied to at least one of the plurality of target atoms; and execute a molecular dynamics simulation for the plurality of atoms using the information regarding the first action force, the information regarding the first additional force, and position information of the plurality of atoms.

### BRIEF DESCRIPTION OF THE DRAWINGS

FIG. 1 is a block diagram illustrating an exemplary hardware configuration of an information processing apparatus according to an embodiment;

FIG. 2 is a diagram illustrating an exemplary functional block in a processor according to an embodiment;

FIG. 3 is a flowchart illustrating an example of a procedure for an MD simulation execution processing according to an embodiment;

FIG. 4 is a diagram illustrating an example of an equilibrated initial structure arranged in a simulation space, a reference atom in a functional group in the initial structure, and a bondable atom, according to an embodiment;

FIG. 5 is a diagram illustrating an example of a search range centered on the position of an identified reference atom and a plurality of atoms around the reference atom, according to an embodiment;

FIG. 6 is a diagram illustrating an example of a plurality of different fixed boost potentials versus an interatomic distance, according to an embodiment;

FIG. 7 is a diagram illustrating an example of a potential energy surface versus an interatomic distance between reference and bondable atoms, and a boost potential, according to an embodiment;

FIG. 8 is a diagram illustrating an example of a plurality of an accumulated boost potential, a potential barrier, and boost application positions versus an interatomic distance, upon setting a time-dependent boost potential as an acceleration condition, according to an embodiment;

FIG. 9 is a diagram illustrating an example of a potential energy surface versus an interatomic distance, and an accumulated boost potential, upon setting a time-dependent boost potential as an acceleration condition, according to an embodiment;

FIG. 10 is a diagram illustrating an example of an acceleration condition that is set for a plurality of atomic pairs with respect to a single reference atom, according to an embodiment;

FIG. 11 is a diagram illustrating an example of additional force corresponding to different fixed boost potentials in FIG. 6, according to an embodiment;

FIG. 12 is a schematic diagram illustrating an overview of additional force in FIG. 5, according to an embodiment;

FIG. 13 is a diagram illustrating an example of the formation of a chemical bonding between a reference atom and a bondable atom, according to an embodiment; and

FIG. 14 is a diagram illustrating an example of a neural network potential and a specific neural network, according to an application example of an embodiment.

### DETAILED DESCRIPTION OF THE PREFERRED EMBODIMENTS

Embodiments are now described in detail with reference to the drawings.

### Embodiment

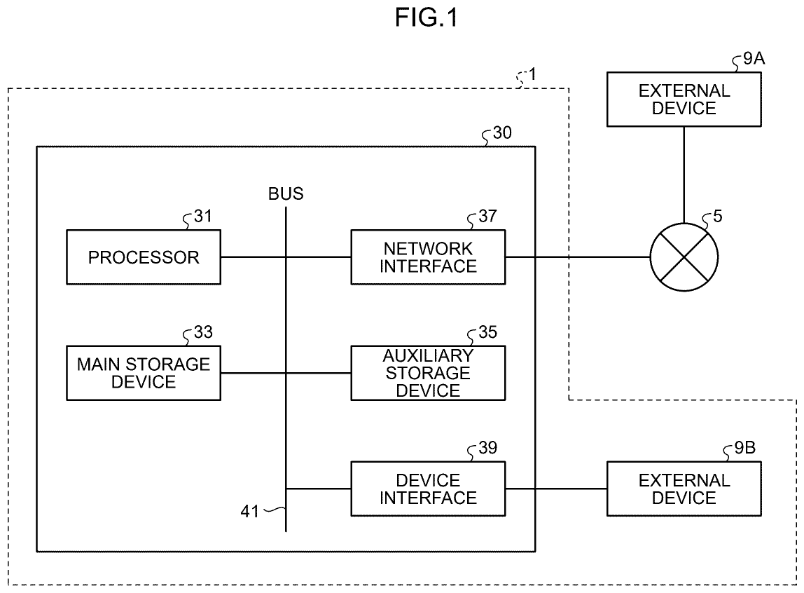

FIG. 1 is a block diagram illustrating an exemplary hardware configuration of an information processing apparatus 1 according to the present embodiment. As illustrated in FIG. 1, the information processing apparatus 1 may be connected to an external device 9A via a communication network 5. Additionally, the information processing apparatus 1 may also include an external device 9B connected via a device interface 39. The information processing apparatus 1 may receive input of a notation indicating the structure of a substance composed of a plurality of atoms entered by a user. The substance may be, for example, a molecule. Moreover, the substance is not limited to a molecule but may also be various crystals or the like.

The notation is, for example, a simplified molecular input line entry system (SMILES) notation entered by a user in relation to the substance. The SMILES notation is, for example, a representation of information regarding a specific molecule (such as information regarding atoms and how they are connected) in accordance with a predefined rule. For example, in the case of methane, the SMILES notation provides information at a granularity level indicating that a single carbon (C) atom is bonded to four hydrogen (H) atoms. Moreover, the notation is not limited to the SMILES notation, but any other known notation may be used as long as it uniquely identifies the substance. Examples of other notations include the SMILES arbitrary target specification (SMARTS) notation. In the following, for the sake of concrete description, the information entered by the user via an input device described later is assumed to be information corresponding to the SMILES notation (hereinafter referred to as SMILES information).

The information processing apparatus 1 includes a computer 30 and the external device 9B connected to the computer 30 via the device interface 39. The computer 30 includes, for example, a processor 31, a main storage device (memory) 33, an auxiliary storage device (memory) 35, a network interface 37, and the device interface 39. The information processing apparatus 1 may be implemented as the computer 30 in which the processor 31, the main storage device 33, the auxiliary storage device 35, the network interface 37, and the device interface 39 are connected via a bus 41.

The computer 30 illustrated in FIG. 1 includes one of each component but may include multiple of the same component. Additionally, although a single computer 30 is illustrated in FIG. 1, the software may be installed across multiple computers, with each of the multiple computers executing the same or different parts of the software. In this case, each computer communicates via the network interface 37 or the like, and the processing may be executed in a distributed computing configuration. In other words, the information processing apparatus 1 in the present embodiment may be configured as a system in which one or more computers execute instructions stored in one or more storage devices to implement various functions described later. Further, the information transmitted from a terminal may be processed by one or more computers provided in the cloud, and the processing result may be transmitted to a terminal such as a display device (display unit) corresponding to the external device 9B.

The various computational operations of the information processing apparatus 1 in the present embodiment may be executed in parallel using one or more processors or using multiple computers via a network. Furthermore, the various computational operations may be distributed to multiple computational operation cores in a processor and executed in parallel processing. Additionally, a portion or the entirety of processing, means, or the like disclosed herein may be executed by at least one of a processor and a storage device provided on the cloud that is communicable with the computer 30 via a network. In this way, the term “various” described in the present embodiment may herein refer to parallel computing, including implementations using one or more computers.

The processor 31 may be an electronic circuit (such as a processing circuit, processing circuitry, central processing unit (CPU), graphics processing unit (GPU), field-programmable gate array (FPGA), or application-specific integrated circuit (ASIC)) that includes a control device and a computational operation device of the computer 30. Additionally, the processor 31 may also be a semiconductor device that includes a dedicated processing circuit. The processor 31 is not limited to an electronic circuit using an electronic logic element and may be implemented by an optical circuit using an optical logic element. Furthermore, the processor 31 may also include a computational operation function based on quantum computing.

The processor 31 is capable of performing computational operation processing based on data and software (programs) received from each device in the internal configuration of the computer 30 and outputting a result obtained by computational operation and a control signal to each unit or device and the like. The processor 31 may control respective components that constitute the computer 30 by executing an operating system (OS) of the computer 30, an application, or other programs.

The information processing apparatus 1 in the present embodiment may be implemented by one or more processors 31. The processor 31 may herein refer to one or more electronic circuits arranged on a single chip or one or more electronic circuits distributed across two or more chips or two or more devices. In the case of using multiple electronic circuits, the respective electronic circuits may communicate with each other via either a wired or wireless connection.

The main storage device 33 is a storage device that stores instructions executed by the processor 31 and various types of data, or the like, and information stored in the main storage device 33 is read by the processor 31. The auxiliary storage device 35 is a storage device distinct from the main storage device 33. Moreover, such storage devices refer to any electronic components or systems capable of storing electronic information, including, but not limited to, semiconductor memory. The semiconductor memory may be either volatile or nonvolatile. The storage device used for saving various types of data employed in the information processing apparatus 1 according to the present embodiment may be implemented by the main storage device 33 or the auxiliary storage device 35, or may be implemented by internal memory built into the processor 31. For example, in the present embodiment, a storage unit may be implemented by the main storage device 33 or the auxiliary storage device 35.

A plurality of processors may be connected (coupled) to a single storage device (memory), or a single processor 31 may be connected to a single storage device (memory). A single processor may be connected (coupled) to a plurality of storage devices (memories). In the case where the information processing apparatus 1 in the present embodiment is configured by at least one storage device (memory) and multiple processors connected (coupled) to at least one storage device (memory), at least one among the multiple processors may include a configuration in which at least one processor is connected (coupled) to the at least one storage device (memory). Additionally, such a configuration may also be implemented by the storage device (memory) and the processor 31 included in the multiple computers. Furthermore, a configuration in which the storage device (memory) is integrated with the processor 31 (e.g., a cache memory including an L1 cache and an L2 cache) may be included.

The network interface 37 is an interface used for connection to the communication network 5 via either a wired or wireless connection. The network interface 37 is acceptable as long as it is an appropriate interface, such as one that conforms to existing communication standards. The network interface 37 may enable information to be exchanged with the external device 9A connected via the communication network 5. Moreover, the communication network 5 may be any of, or a combination of, a wide area network (WAN), a local area network (LAN), a personal area network (PAN), and the like, as long as the communication network 5 enables the exchange of information between the computer 30 and the external device 9A. Examples of a WAN include the Internet, Examples of a LAN include IEEE 802.11 or Ethernet (registered trademark), and Examples of a PAN include Bluetooth (registered trademark) or near-field communication (NFC).

The device interface 39 is an interface such as an output device, an input device, and a universal serial bus (USB) that enables direct connection to the external device 9B. Moreover, the output device may include a speaker or the like that outputs sound or similar.

The external device 9A is a device that is connected to the computer 30 via a network. The external device 9B is a device that is directly connected to the computer 30.

The external device 9A or the external device 9B may be, for example, an input device (input unit). Examples of the input device may include a device such as a camera, a microphone, a motion capture device, various sensors, a keyboard, a mouse, or a touch panel, and provides the acquired information to the computer 30. Additionally, the external device 9A or the external device 9B may also be a device or the like equipped with an input unit, memory, and a processor, such as a personal computer, a tablet terminal, or a smartphone.

Further, the external device 9A or the external device 9B may also be, for example, an output device (output unit). Examples of the output device may include a display device (display unit) such as a liquid crystal display (LCD), a cathode ray tube (CRT), a plasma display panel (PDP), or an organic electro-luminescence (EL) panel, or may include a speaker or the like that outputs sound or similar. Additionally, the external device 9A or the external device 9B may be a device or the like that includes an output device, memory, and a processor, such as a personal computer, a tablet terminal, or a smartphone.

Further, the external device 9A or the external device 9B may be a storage device (memory). For example, the external device 9A could be network storage or the like, and the external device 9B could be storage such as an HDD.

Further, the external device 9A or the external device 9B may be a device that has some of the functions of the components of the information processing apparatus 1 in the present embodiment. In other words, the computer 30 may transmit or receive a portion or the entirety of the result obtained by the processing in the external device 9A or the external device 9B.

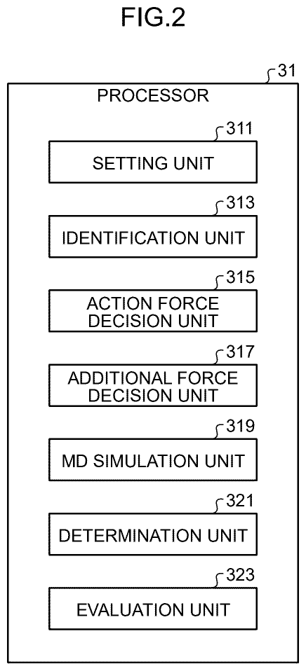

FIG. 2 is a diagram illustrating an exemplary functional block implemented by one or more processors 31. The processor 31 includes, for example, a setting unit 311, an identification unit 313, an action force decision unit 315, an additional force decision unit 317, a molecular dynamics (MD) simulation unit 319, a determination unit 321, and an evaluation unit 323, as functions implemented by the processor 31. The functions implemented by the setting unit 311, the identification unit 313, the action force decision unit 315, the additional force decision unit 317, the MD simulation unit 319, the determination unit 321, and the evaluation unit 323 are each stored as a program in, for example, the main storage device 33 or the auxiliary storage device 35. The processor 31 is capable of reading and executing each program stored in the main storage device 33 or the auxiliary storage device 35 to implement the functions regarding the setting unit 311, the identification unit 313, the action force decision unit 315, the additional force decision unit 317, the MD simulation unit 319, the determination unit 321, and the evaluation unit 323.

The setting unit 311 sets various conditions regarding the MD simulation (hereinafter referred to as a simulation condition). For example, the setting unit 311 sets the simulation condition based on a user instruction received via an input device or by reading the simulation condition from the main storage device 33 and/or the auxiliary storage device 35. The setting unit 311 stores the set simulation condition in the main storage device 33 and/or the auxiliary storage device 35.

Examples of the simulation condition include a search condition regarding the searching for a plurality of target atoms subject to chemical bonding, an acceleration condition regarding the acceleration of chemical bonding, the temperature and pressure within a virtual space (simulation space) where the MD simulation is performed, and the composition within the virtual space where the MD simulation is performed (e.g., such as types of monomer, types of solvent, types of starting substance, or composition ratio).

Examples of the search condition include a search range regarding the search for the plurality of target atoms. The search range may be set, for example, by the user or may be set in advance. The search range corresponds to, for example, a region between the minimum and maximum distances from a reference atom that is a reference for chemical bonding among the plurality of target atoms. In this case, the search condition corresponds to two distances of the minimum distance and the maximum distance.

In the following, for a more specific description, the plurality of target atoms is assumed to be a pair (atomic pair) of a reference atom and a single atom that is capable of forming a chemical bonding with the reference atom (hereinafter referred to as a bondable atom). The bondable atom corresponds to an atom that has the potential to react with the reference atom. In other words, the bondable atom corresponds to an atom that is more likely to react with the reference atom. Moreover, the case where the plurality of target atoms has a reference atom and a plurality of atoms that are bondable to the reference atom will be described as appropriate.

The acceleration condition is, for example, a condition that defines a bulk energy (hereinafter referred to as boost potential) that promotes chemical bonding between multiple target atoms. For example, in the case where a temporally fixed boost potential (hereinafter referred to as fixed boost potential) is applied to a bondable atom, the acceleration condition corresponds to a parameter that defines the fixed boost potential. The parameter that defines the fixed boost potential corresponds to the magnitude of the energy (potential) and the range affected by the energy (potential). Specifically, in the case where the fixed boost potential is expressed by a Gaussian function, the magnitude of the boost potential corresponds to the maximum value of the Gaussian function, and the range affected by the boost potential corresponds to the half-width (full-width at half maximum or half-width at half maximum) of the Gaussian function or the like. In other words, the fixed boost potential corresponds to a boost potential that is independent of time. Moreover, the shape of the boost potential is not limited to a Gaussian function that defines a Gaussian potential, and any function such as a Morse potential or a Lennard-Jones potential may be used, or may be defined by any function expressed by a neural network or the like.

Further, in the case where the boost potential applied to the atomic pair is a time-dependent boost potential (hereinafter referred to as a time-dependent boost potential), the acceleration condition corresponds to a parameter that defines the time-dependent boost potential applied per unit time. The parameter that defines the time-dependent boost potential includes the magnitude of the energy (potential), the range affected by the energy (potential) applied per unit time, the frequency at which the energy (potential) is applied, or the like. Additionally, the shape of the boost potential may be defined by a shape obtained by inverting a probability distribution that indicates the existence probability depending on the coordinates of the bondable atoms, in accordance with a Boltzmann distribution or the like.

In the case where the time-dependent boost potential is a plurality of target atoms (an atomic group), that is, the case where the plurality of target atoms has a reference atom and a plurality of bondable atoms, if the boost potential applied to an atomic group constituted by the plurality of bondable atoms is a time-dependent boost potential, the acceleration condition corresponds to the magnitude of the boost potential, the range affected by the boost potential, the shape of an activation function, the degree of weighting for each of multiple group variables, and the like. The shape of the activation function and the degree of weighting will be described later.

The setting unit 311, prior to the execution of the MD simulation, arranges a plurality of atoms (molecules) in a virtual space in which the MD simulation is to be executed. This arrangement of the plurality of atoms in the virtual space causes the setting unit 311 to set an initial structure of the simulation target. The setting unit 311 causes the set initial structure to be stored in the main storage device 33 and/or the auxiliary storage device 35.

The setting unit 311 performs equilibration on the initial structure. Specifically, the setting unit 311 moves the positions of a plurality of molecules and/or a plurality of atoms in the virtual space so that the state of the plurality of molecules included in the initial structure and/or the state of the plurality of atoms included in the initial structure reaches mechanically and thermally stable (hereinafter referred to as a metastable state) depending on the temperature, pressure, and other conditions, among the simulation conditions set for the virtual space having the initial structure. Since known techniques can be applied for the equilibration processing, further description is omitted. The setting unit 311 causes the position of the plurality of molecules and/or the plurality of atoms after equilibration in the simulation space (hereinafter referred to as an initial position) to be stored in the main storage device 33 and/or the auxiliary storage device 35.

The identification unit 313 identifies a plurality of target atoms subject to chemical bonding among the plurality of atoms. Specifically, the identification unit 313 identifies the plurality of target atoms subject to chemical bonding among the plurality of atoms arranged in the virtual space regarding the MD simulation. The plurality of target atoms corresponds to atoms that are more likely to react chemically. Identifying the plurality of target atoms corresponds to listing the atoms that are more likely to react chemically. For example, in response to a user instruction via the input interface, the identification unit 313 identifies a plurality of atoms that is more likely to chemically bond (react chemically) among multiple atoms arranged in the virtual space. The atoms that are more likely to chemically bond (react chemically) are, for example, atoms contained in functional groups such as radicals and vinyl carbon, and correspond to a reference atom that serves as the reference for chemical bonding. For example, the identification unit 313 identifies the sites that accelerate bond formation/decomposition in functional group units in accordance with the user instruction provided via the input interface.

The identification unit 313 searches for atoms included in the search range in the virtual space regarding the MD simulation, with each of the identified atoms (reference atoms) as the center. The identification unit 313 identifies an atom included in the search range of each of the reference atoms as the bondable atom. Moreover, for each of the multiple reference atoms, an atom located closer than the minimum distance of the search range is more liable to already be chemically bonded to the reference atom, so the aforementioned atom may be excluded from the search for bondable atoms. The identification unit 313 associates the bondable atom identified in the search with the reference atom that serves as the reference for the search. In this way, the identification unit 313 identifies an atomic pair of the reference atom and the bondable atom. The identification unit 313 causes the identified multiple atomic pairs, that is, a plurality of target atoms, to be stored in the main storage device 33 and/or the auxiliary storage device 35. In this case, the reference atom and the bondable atom are associated using an index that distinguishes the atoms and are stored in the main storage device 33 and/or the auxiliary storage device 35.

If the determination unit 321, which will be described later, determines that the chemical bonding is formed and the sum of the boost potentials is subsequently initialized, the identification unit 313 updates attribute information of the atom with which the chemical bonding is formed, based on the functional groups regarding the plurality of atoms with which the chemical bonding is formed. The attribute information of the atom with which the chemical bonding is formed is information regarding an attribute that indicates whether the plurality of atoms contained in the molecule with which the chemical bonding is determined to be formed is likely to react chemically. This updates the reference atom regarding the formed chemical bonding. Subsequently, the identification unit 313 applies the center of the search range to the position of the updated reference atom to identify at least one bondable atom that is capable of forming a chemical bonding with the reference atom. In this way, the identification unit 313 updates the plurality of target atoms. The update of the reference atom in the plurality of atoms with which the chemical bonding is formed is executed using a preconfigured known program or the like.

Further, if the determination unit 321 described later determines that a chemical bonding is not formed and also determines that the MD simulation unit 319 described later executes the MD simulation for a predetermined time or a predetermined number of times, the identification unit 313 identifies a reference atom (or an index of the reference atom) that is not related to the application of the boost potential. In this case, the processing for the identified reference atom is executed by the additional force decision unit 317 described later. The predetermined time and the predetermined number of times are preset by the setting unit 311 in accordance with an instruction from a user or the like, and are stored in the main storage device 33 and/or the auxiliary storage device 35.

The action force decision unit 315 acquires information regarding a first action force (hereinafter referred to as action force or first action force) acting on each of multiple atoms, which is generated by inputting an atomic structure of the multiple atoms into a trained neural network potential (hereinafter referred to as NNP). For example, the action force decision unit 315 inputs the atomic structure of the plurality of atoms into the trained neural network and generates information regarding the first action force acting on each of the plurality of atoms. The information regarding the first action force conceptually includes, for example, the value of the first action force itself and/or information necessary for deciding the first action force. Specifically, the action force decision unit 315 decides the first action force of each of the multiple atoms by inputting the position of the atoms arranged in the virtual space and the information regarding the types of the arranged atoms (atomic structure) into the trained NNP. The atomic structure includes, for example, information regarding the type of the plurality of atoms and the position information of the plurality of atoms. The position information of the atom includes, for example, the coordinates of the atom. Moreover, the position information of the atom may be in any format as long as the information regarding the position represents information regarding the position of the atom. The NNP is implemented, for example, using a trained graph neural network with high versatility that is capable of generating a highly accurate energy value and force acting on each of the multiple atoms for various atomic structures. Since a known neural network can be applied as the trained graph neural network, further description is omitted. The action force decision unit 315 causes the multiple forces acting on the respective multiple atoms arranged in the virtual space to be stored in the main storage device 33 and/or the auxiliary storage device 35.

Further, if the determination unit 321 described later determines that a chemical bonding is not formed and that the MD simulation is not executed for a predetermined time or a predetermined number of times, then the action force decision unit 315 acquires information regarding a second action force acting on each of the plurality of atoms, which is generated by inputting the atomic structure of the plurality of atoms into the NNP. The information regarding the second action force conceptually includes, for example, the value of the second action force itself and/or information necessary to decide the second action force. For example, the action force decision unit 315 decides the second action force acting on each of the multiple atoms by inputting the position of the plurality of target atoms and the type of the plurality of atoms after (immediately after) the execution of the MD simulation into the NNP. In other words, the action force decision unit 315 sequentially calculates the action force for each of the multiple atoms moved by the MD simulation each time the MD simulation is executed. The action force decision unit 315 may be referred to as an action force acquisition unit.

The additional force decision unit 317 acquires information regarding a first additional force to be applied to at least one of the plurality of target atoms. Specifically, the additional force decision unit 317 applies a boost potential to each of the plurality of target atoms using the coordinates of each of the plurality of target atoms. For example, the additional force decision unit 317 applies a boost potential depending on the position of a reference atom having an index selected from the plurality of reference atoms and the position of a bondable atom corresponding to the selected reference atom. For example, in the case where the boost potential is expressed as a Gaussian function and the plurality of target atoms form an atomic pair, the boost potential is applied to the position of the bondable atom so that the maximum value of the Gaussian function is located at the position of the reference atom and the position of the bondable atom. Moreover, the additional force decision unit 317 may apply the additional force to only one of the target atoms, not just to both of the target atoms. The information regarding the first additional force conceptually includes, for example, the value of the first additional force itself and/or information necessary for deciding the first additional force.

The additional force decision unit 317 decides a force to be applied to each of the plurality of target atoms (hereinafter referred to as the additional force or the first additional force), based on the coordinates of the plurality of target atoms and the boost potential. For example, the additional force decision unit 317 decides information regarding the first additional force based on the position information of the plurality of target atoms. Specifically, the additional force decision unit 317 decides the information regarding the first additional force based on the position information of the plurality of target atoms and the boost potential. The information regarding the first additional force is not limited to the value of the first additional force itself, but may also include information necessary for deciding the first additional force. The information regarding the first additional force may be information regarding a value to be multiplied by the first action force. Specifically, in the case where the first action force created by the NNP is in the desired direction and/or in the direction in which the reaction proceeds, the information regarding the first additional force may be expressed not by vector addition of the additional force but by multiplying the first action force decided by the NNP, such as multiplying it by 1.5. Additionally, the information regarding the first additional force may also be information that the value of the first additional force is zero (no additional force is applied). For example, in the case where the distance between the target atoms is less than a predetermined threshold, the additional force decision unit 317 decides, as the information regarding the first additional force, that the first additional force is not to be applied to the plurality of target atoms.

The calculation of the additional force applied to the plurality of target atoms (e.g., atomic pairs) from the boost potential can be implemented using a known technique, such as analytical calculation based on the boost potential and the position of the plurality of target atoms (e.g., atomic pairs), so further description is omitted. The additional force decision unit 317 causes the additional force applied to each of the plurality of target atoms (e.g., atomic pairs) to be stored in the main storage device 33 and/or the auxiliary storage device 35.

Moreover, the additional force decision unit 317 may decide the information regarding the first additional force without reliance on the boost potential. For example, the first additional force may be a fixed value. In this case, the additional force decision unit 317 may decide the fixed value based on position information between atoms and distance information between atoms. Additionally, the additional force decision unit 317 may also be configured to add the fixed value each time the MD simulation is repeatedly executed.

Further, if the determination unit 321 determines that a chemical bonding is not formed and also determines that the MD simulation is not performed for a predetermined time or a predetermined number of times, then the additional force decision unit 317 may further apply a boost potential to the position of the atomic pair before the movement of the atomic pair (e.g., the position of the bondable atom). In this case, the additional force decision unit 317 applies a boost potential again to the position of the bondable atom. In other words, if it is continuously determined that a chemical bonding is not formed in multiple MD simulations for a predetermined time or a predetermined number of times, then the additional force decision unit 317 accumulates the boost potential applied for a predetermined number of times depending on the past position of the paired atoms. Moreover, the accumulation of the boost potential may be performed by the setting unit 311. In other words, if the determination unit 321 determines that a chemical bonding is not formed between the target atoms, the MD simulation unit 319 re-executes the MD simulation using the information regarding the first additional force. The further use of the information regarding the first additional force corresponds to, for example, the accumulation of the boost potential. Moreover, the meaning of “executing an MD simulation using XX and YY” is not limited to executing a simulation using XX and YY directly, but includes executing a simulation using ZZ generated based on XX and YY.

For example, if a chemical bonding is determined not to be formed, the additional force decision unit 317 calculates a sum of the boost potential regarding the first additional force decided before the execution of the MD simulation and the boost potential applied to the coordinates of the plurality of target atoms (e.g., atomic pairs) after the execution of the MD simulation. In this way, the additional force decision unit 317 decides the additional force (a second additional force) to be applied to each of the plurality of target atoms based on the coordinates of the plurality of target atoms after the execution of the MD simulation and the sum of the boost potential. In other words, the additional force decision unit 317 acquires information regarding the second additional force to be applied to at least one of the plurality of target atoms. The information regarding the second additional force conceptually includes, for example, the value of the second additional force itself and/or information necessary to decide the second additional force.

Moreover, in the case where the distance between the atomic pairs, that is, the distance between the reference atom and the bondable atom, is less than a predetermined distance (e.g., the minimum distance of the search range, which may be referred to as a predetermined threshold), the additional force decision unit 317 may not apply the boost potential to the position of the bondable atom. Furthermore, the additional force decision unit 317 may also set a potential wall (potential barrier) that is greater than the maximum value of the boost potential at a position that is the maximum distance away from the reference atom. The additional force decision unit 317 may also be referred to as an additional force acquisition unit.

The MD simulation unit 319 executes a molecular dynamics simulation for multiple atoms using the information regarding the first action force, the information regarding the first additional force, and the position information of the multiple atoms. Specifically, before the execution of the MD simulation, in this case, the MD simulation unit 319 calculates the sum of the action force decided by the action force decision unit 315 and the additional force decided by the additional force decision unit 317. For example, the MD simulation unit 319 calculates the sum of the first action force and the first additional force. Subsequently, the MD simulation unit 319 uses the sum of the first action force and the first additional force, as well as the position of multiple atoms, to execute an MD simulation for the multiple atoms over preset minute time. In other words, the MD simulation unit 319 executes an MD simulation for multiple atoms using the sum of the first action force and the first additional force, as well as the position information of the multiple atoms.

Further, after the initialization of the boost potential, the MD simulation unit 319 calculates the sum of the second action force and the second additional force. Subsequently, the MD simulation unit 319 executes the MD simulation for the multiple atoms over preset minute time using the sum of the second action force and the second additional force, as well as the position of the multiple atoms. This allows the MD simulation unit 319 to re-execute the MD simulation for the multiple atoms using the information regarding the second action force, the information regarding the second additional force, and the position information of the multiple atoms after the MD simulation. The processing procedure for the MD simulation may employ known techniques, so further description is omitted. The MD simulation unit 319 causes the position of the multiple atoms after the movement as a result of the MD simulation to be stored in the main storage device 33 and/or the auxiliary storage device 35, along with the measurement time of the MD simulation.

The determination unit 321 determines the presence or absence of chemical bonding formation based on the position information of the plurality of target atoms after the execution of the MD simulation. For example, the determination unit 321 reads a determination condition (hereinafter referred to as a bond determination condition) regarding the determination of the presence or absence of a chemical bonding from the main storage device 33 and/or the auxiliary storage device 35. Examples of the bond determination condition include a correspondence table indicating the interatomic distance (hereinafter referred to as bond determination distance) for the type of bond such as a covalent bond. Moreover, the bond determination distance may be the distance of a bond set depending on the type of bond (e.g., a covalent bond distance for a covalent bond) plus a margin of a predetermined length. The margin may be preset by a user instruction via an input interface and/or by the setting unit 311. The correspondence table is preset using a chemical database or the like and stored in the main storage device 33 and/or the auxiliary storage device 35.

The determination unit 321 calculates the distance between the reference atom and the bondable atom (hereinafter referred to as the interatomic distance). Additionally, the determination unit 321 also identifies the type of bond between the reference atom and the bondable atom. The identification of the type of bond is based on known techniques for identifying the type of functional group formed by the reference atom and the bondable atom. Thus, further description is omitted. The determination unit 321 identifies the bond determination distance from the correspondence table by checking the identified type of bond against the correspondence table. Subsequently, the determination unit 321 compares the interatomic distance with the bond determination distance to determine the presence or absence of a chemical bonding between the reference atom and the bondable atom. Specifically, if the interatomic distance is equal to or less than the bond determination distance, the determination unit 321 determines that the reference atom and the bondable atom are chemically bonded. On the other hand, if the interatomic distance exceeds the bond determination distance, the determination unit 321 determines that the reference atom and the bondable atom are not chemically bonded.

Further, if a chemical bonding is determined not to be formed, the determination unit 321 determines whether the MD simulation is executed for a predetermined time or a predetermined number of times. For example, if the sum of the minute time regarding the execution of the MD simulation is less than the predetermined time, the boost potential is applied again to the bondable atom to which the boost potential is applied before the execution of the MD simulation. Additionally, if a chemical bonding is determined not to be formed and the sum of the minute time (the sum of the execution time of the MD simulation regarding the reference atom) is equal to or more than the predetermined time, the determination unit 321 initializes (sets to zero) the sum of the boost potentials applied to the plurality of target atoms. Moreover, the initialization of the sum of the boost potentials may be executed by the identification unit 313 or the additional force decision unit 317.

Following the update of the attribute information by the identification unit 313, the determination unit 321 determines whether the number of atoms included in the specified atomic attribute (i.e., the updated attribute) is equal to or less than a certain number. The certain number may be preset or may be set by the setting unit 311. For example, in the case where the specified atomic attribute is a vinyl group and there are 100 carbon atoms in the vinyl group initially (before execution of the MD simulation), the number of vinyl groups gradually decreases due to the chemical bonding formation during the MD simulation. If the number of carbon atoms in the vinyl group reaches zero, the reaction can no longer proceed, so the MD simulation proceeds to the next step, for example, processing executed by the identification unit 313 to identify the plurality of target atoms for chemical bonding. The certain number is, for example, zero as mentioned above, but is not limited thereto, and for example, the MD simulation may be terminated in the case where the number of atoms to be attributed has reacted 50% from the initial value (in the above example, 50 carbon atoms in the vinyl group). In other words, the certain number is the number of atoms involved in the chemical reaction within the functional group regarding the chemical reaction, the ratio of the number of reacted atoms to the total number of atoms regarding the chemical reaction, or the like. If the number of atoms included in the specified atomic attribute is equal to or less than a certain number, an evaluation of the molecule generated by the chemical reaction is performed.

The evaluation unit 323 evaluates the physical properties of the molecule (e.g., a macromolecule such as a polymer) generated by the MD simulation. Examples of the physical properties include thermal properties (e.g., heat resistance) and mechanical properties (e.g., tensile properties) of the generated molecule. Additionally, the evaluation unit 323 may also evaluate the distribution of the length of the polymer. Since known techniques can be applied for the evaluation of the physical properties of the molecule, further description is omitted. The evaluation unit 323 causes the evaluated physical properties of the molecule to be stored in the main storage device 33 and/or the auxiliary storage device 35. Moreover, the evaluation unit 323 may cause the evaluated molecular properties to be displayed on a display or the like.

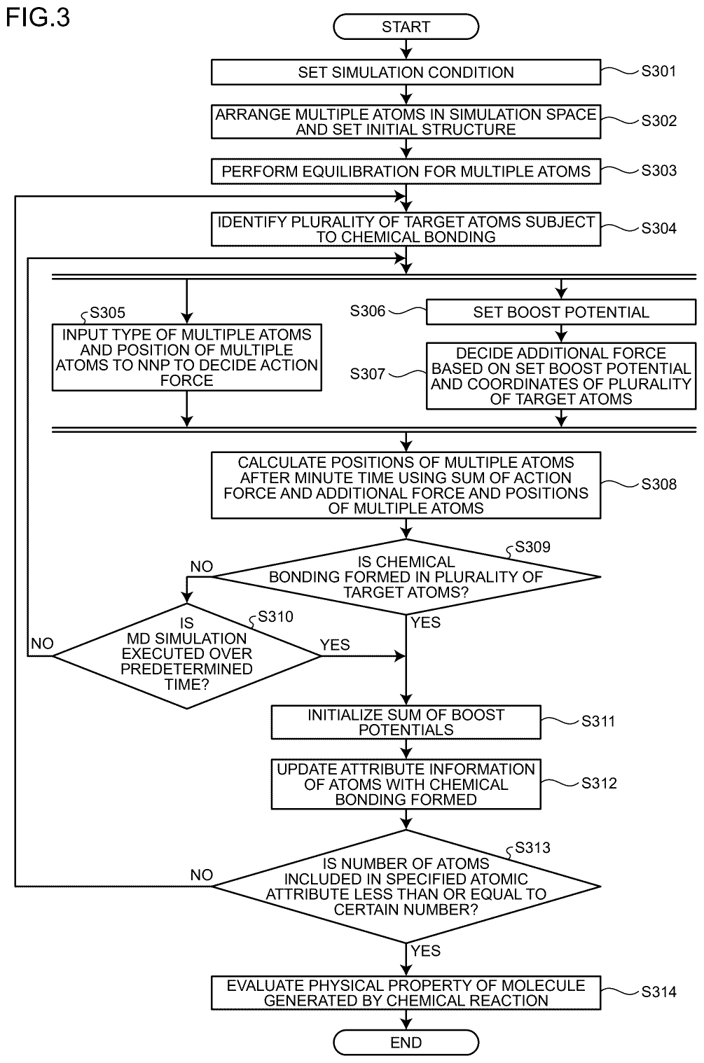

In the above, the configuration of the information processing apparatus 1 is described. The procedure of the MD simulation processing executed by the information processing apparatus 1 (hereinafter referred to as MD simulation execution processing) is now described with reference to FIG. 3.

FIG. 3 is a flowchart illustrating an example of the procedure for executing MD simulation processing.

### Md Simulation Execution Processing

### Step S301

The setting unit 311 sets a simulation condition. The setting of the simulation condition is not limited to reading the simulation condition from the main storage device 33 and/or the auxiliary storage device 35, but may also be performed by the user instruction via the input interface. In this case, the simulation condition may be adjusted as appropriate by the user.

### Step S302

The setting unit 311 arranges a plurality of atoms in the simulation space (simulation cell, simulation box) and sets an initial structure. For example, the setting unit 311 sets the initial structure by randomly arranging the multiple atoms in the simulation space. The simulation space can be optionally set as a rectangular parallelepiped, cube, sphere, or other shapes.

### Step S303

The setting unit 311 performs equilibration for the multiple atoms. For example, the setting unit 311 achieves equilibration by moving multiple atoms arranged in the simulation space so that the multiple atoms are in a metastable state.

### Step S304

The identification unit 313 identifies a plurality of target atoms subject to chemical bonding among the multiple atoms arranged in the simulation space. In other words, the identification unit 313 identifies an atom that is more likely to react chemically as a reference atom among the multiple atoms. For example, sites that accelerate a chemical reaction (multiple atoms) such as bond formation or decomposition are specified in functional group units based on a user instruction via the input interface. In this case, the identification unit 313 identifies a reference atom that serves as a reference for the chemical reaction in the specified functional group.

Moreover, the identification unit 313 may include processing for excluding pairs of atoms that have already formed chemical bonding in identifying a plurality of target atoms subject to chemical bonding. The most typical processing executed by the identification unit 313 in the above step is to prepare a list of atoms in advance and update the list as a bond is formed and broken. For example, in atomic group A={a1, a2, a3, . . . } and atomic group B={b1, b2, b3, . . . }, if a reaction occurs between two atoms included in different atomic groups (e.g., a1+b2→a1−b2), the relevant atoms (a1 and a2) regarding the reaction are deleted from atomic groups A and B upon the reaction occurs. In this event, the identification unit 313 may determine whether a chemical bonding occurs between the atoms based on the distance between the atoms.

Moreover, the identification of the target atom is not limited to the above. For example, the identification unit 313 may identify attribute information of the atom based on bond information generated using the distance between atoms and identify the target atom using a general reaction rule. For example, based on input using the SMILES notation, the identification unit 313 is capable of identifying a carbon radical that has only three bonds and one unpaired electron. Similarly, based on input using the SMILES notation, the identification unit 313 is capable of identifying a vinyl carbon that has a carbon atom connected by a double bond. The general reaction rule corresponds to, for example, the knowledge that a carbon radical reacts with a vinyl carbon. Storing such a main reaction rule in the main storage device 33 and/or the auxiliary storage device 35 in advance enables the identification unit 313 to identify the target atom by comparing the rule with the attribute information of the atom.

The identification unit 313 identifies, as a bondable atom, the atom closest to the reference atom among the multiple atoms included in the search range centered on the position of the identified reference atom. Moreover, the identification unit 313 may identify, as a bondable atom, a predetermined number of multiple atoms included in the search range in order of proximity of distance from the reference atom.

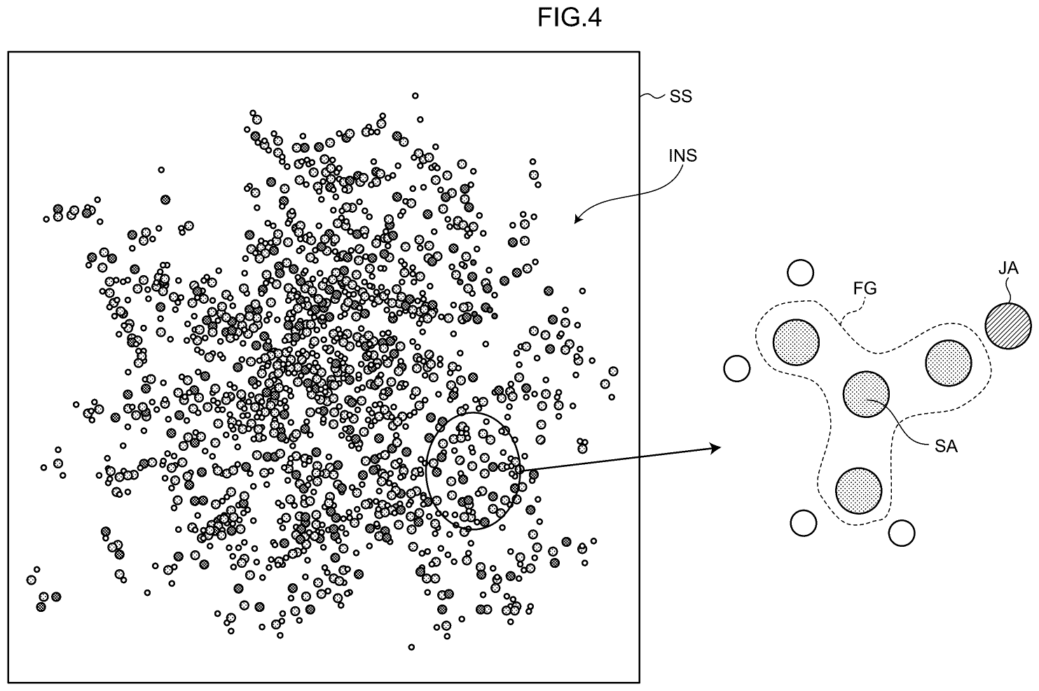

FIG. 4 is a diagram illustrating an example of an initial structure INS arranged in a simulation space SS and equilibrated, as well as an example of a reference atom SA and a bondable atom JA in a functional group FG in the initial structure INS. As illustrated in FIG. 4, the identification unit 313 identifies an atomic pair of a reference atom SA and a bondable atom JA that are subject to chemical bonding among the multiple atoms included in the initial structure INS arranged in the simulation space SS.

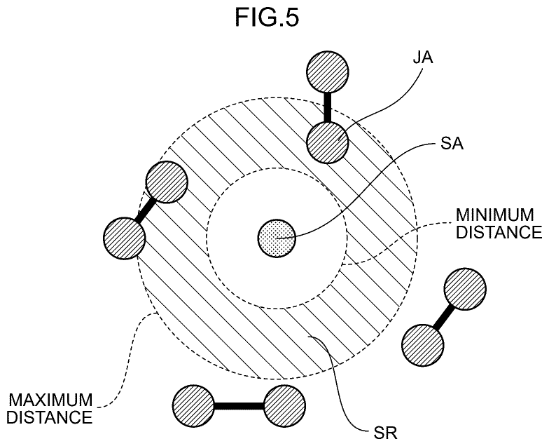

FIG. 5 illustrates an example of a search range SR centered on the position of the identified reference atom SA and a plurality of atoms around the reference atom JA. For example, as illustrated in FIG. 5, the identification unit 313 identifies, as the bondable atom JA, an atom included in the search range SR and closest to the reference atom SA. The identification unit 313 identifies a plurality of atomic pairs corresponding to a plurality of target atoms by executing the search illustrated in FIG. 5 for each of the multiple identified reference atoms.

### Step S305

The action force decision unit 315 inputs information regarding the type of the multiple atoms and the position of the multiple atoms (atomic structure) into the NNP. As a result, the action force decision unit 315 decides the action force (first action force) based on the output from the NNP by position differentiation using backward processing. Moreover, in deciding the action force, an NNP configured to directly output the force from the output layer without using backward processing may be used. The action force decision unit 315 causes the decided action force to be stored in the main storage device 33 and/or the auxiliary storage device 35. For example, if a chemical bonding is not formed with respect to the identified reference atom and the execution of the MD simulation is not continued for a predetermined time duration, the action force decision unit 315 updates the action force by inputting the type of the multiple atoms and the position of the multiple atoms moved during the MD simulation into the NNP every time the MD simulation is executed.

Further, for example, in the case where a chemical bonding is formed with respect to the identified reference atom and the number of atoms included in the specified atomic attribute is not equal to or less than a certain number, the action force decision unit 315 inputs the type of the multiple atoms and the position of the multiple atoms into the NNP to decide an action force (second action force) by position differentiation using backward processing. The action force decision unit 315 causes the decided action force to be stored in the main storage device 33 and/or the auxiliary storage device 35.

### Step S306

The setting unit 311 sets a boost potential. For example, in the case where a fixed boost potential expressed by a Gaussian function is set as an acceleration condition, the setting unit 311 sets the maximum value of the Gaussian function corresponding to the maximum energy of the boost potential and the half-width of the Gaussian function corresponding to the range affected by the energy. The setting unit 311 sets the position of the fixed boost potential based on the positions of the reference atom and the bondable atom in the atomic pair. The setting unit 311 causes the set fixed boost potential and the position to which the fixed boost potential is applied to be stored in the main storage device 33 and/or the auxiliary storage device 35. In the case where the fixed boost potential is set, the boost potential remains constant in the repetition of the MD simulation for the identified reference atom. In this case, if the reference atom is changed, the fixed boost potential is set again for the changed reference atom.

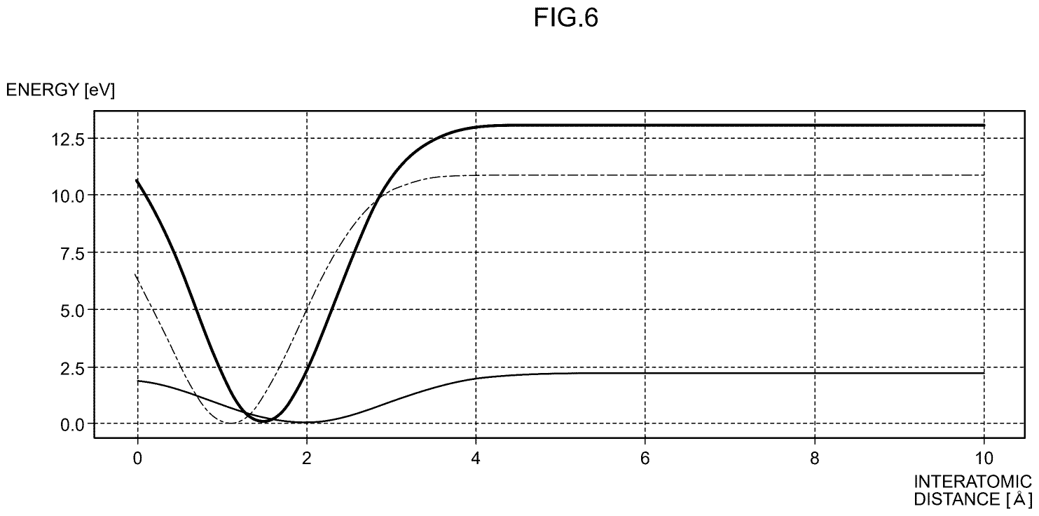

FIG. 6 is a diagram illustrating an example of multiple different fixed boost potentials for the interatomic distance. In FIG. 6, three fixed boost potentials are illustrated, but the number of the fixed boost potentials is not limited to this example. The shape of the fixed boost potential can be set optionally depending on the setting of the parameter that defines the fixed boost potential. The fixed boost potential Eᵣₑₛₜ is defined, for example, by the following Formula (1).

$${E}_{rest} = {F}_{1} \{ 1 - {e}^{- {{F}_{2} ( {R}_{ij} - {R}_{12} )}^{2}} \} \qquad ( 1 )$$

In Formula (1), F₁ on the right side represents the intensity of the boost potential. Additionally, in Formula (1), F₂ on the right side represents the distance affected by the boost potential. In addition, in Formula (1), R₁₂ on the right side represents the equilibrium position between the reference atom and the bondable atom. Furthermore, in Formula (1), Rᵢⱼ on the right side represents the distance between the reference atom and the bondable atom in the equilibrated initial structure INS.

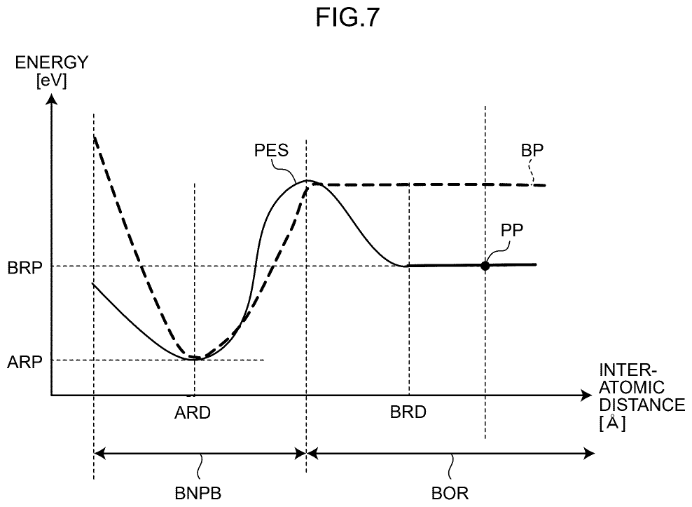

FIG. 7 illustrates an example of a potential energy surface (PES) for the interatomic distance between the reference atom and the bondable atom (referred to as a reaction coordinate or collective variable), and a boost potential BP. The potential energy surface PES represents the distribution of potential energy with respect to the reaction coordinate or collective variable (e.g., a function of energy with respect to the interatomic distance). As illustrated in FIG. 7, the potential energy surface PES corresponding to the interatomic distance after the chemical reaction (hereinafter referred to as a post-reaction position) ARD corresponds to the potential energy after the chemical reaction ARP and reaches a minimum value. On the other hand, as illustrated in FIG. 7, the potential energy surface PES corresponding to the interatomic distance before the chemical reaction (hereinafter referred to as a pre-reaction position) BRD corresponds to the potential energy before the chemical reaction BRP and reaches a minimum value.

As illustrated in FIG. 7, the boost potential BP corresponds to, for example, the potential energy added to the minimum value of the potential energy BRP. For example, the setting unit 311 sets the boost potential BP such that the maximum value of the boost potential BP is located at the pre-reaction position BRD corresponding to the minimum value BRP of the potential energy surface PES. Moreover, the setting position of the maximum value of the boost potential BP is not limited to the pre-reaction position BRD. For example, the boost potential BP may be set such that the maximum value of the boost potential BP is located at a position that is a predetermined distance away from the pre-reaction position BRD.

Further, the setting unit 311 may set a potential barrier at the position of the maximum distance of the search range SR for the reference atom SA, in addition to the boost potential BP. Furthermore, the setting unit 311 may set the boost potential BP in advance so that the boost potential BP is not applied to a position less than the minimum distance of the search range SR for the reference atom SA (hereinafter referred to as a boost-unavailable range).

Further, in the case where a time-dependent boost potential is set as the acceleration condition, the setting unit 311 sets the maximum value of the Gaussian function, the half-width of the Gaussian function, and the frequency of applying the time-dependent boost potential. The frequency can be set optionally, such as per unit time or per number of executions of the MD simulation. The setting unit 311 sets the boost potential applied to the bondable atom corresponding to the identified reference atom, for example, by accumulating the boost potential each time the MD simulation is executed (e.g., the sum of Gaussian potentials corresponding to the number of times the MD simulation is executed). The setting unit 311 causes the accumulated time-dependent boost potential to be stored in the main storage device 33 and/or the auxiliary storage device 35. Moreover, the accumulation of the time-dependent boost potential may be executed in step S307 described later. Furthermore, in this step, the accumulation of the time-dependent boost potential may also be performed by the additional force decision unit 317.

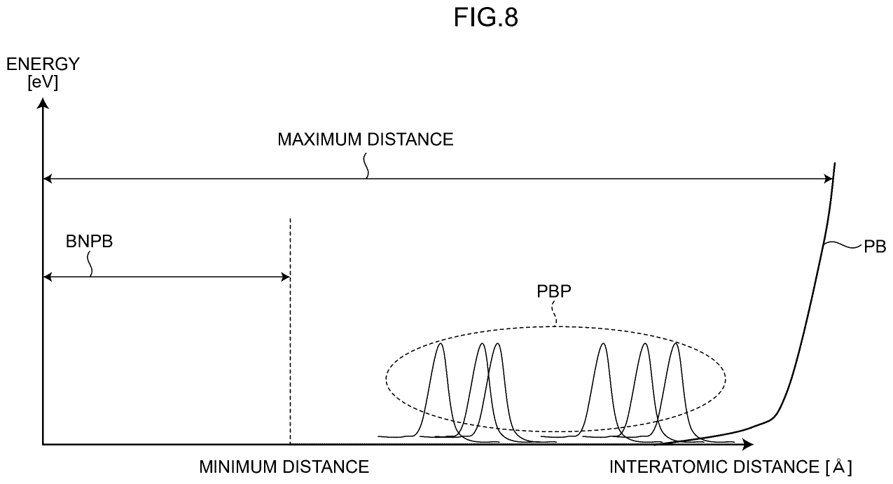

FIG. 8 is a diagram illustrating an example of the multiple accumulated boost potentials PBP, the potential barrier PB, and the boost addition position with respect to interatomic distance in the case where the time-dependent boost potential is set as the acceleration condition. As illustrated in FIG. 8, the multiple time-dependent boost potentials are set, for example, for each MD simulation depending on the set frequency. Additionally, as illustrated in FIG. 8, the potential barrier PB is set, for example, at the end of the maximum distance in the search range SR to prevent the bondable atom from moving away from the reference atom. Furthermore, as illustrated in FIG. 8, a boost-unavailable (boost non-potential) range BNPB is set at the end of the minimum distance in the search range SR. The maximum value of the interatomic distance in the boost-unavailable range BNPB (the minimum distance in FIG. 8) corresponds to the cutoff of the boost potential.

Moreover, as illustrated in FIG. 7, in the case where the interatomic distance is located in a predetermined range away from the potential barrier (hereinafter referred to as an outside-barrier range, BOR), the setting unit 311 may add the boost potential BP to the potential energy surface PES. In other words, the setting unit 311 may fail to add the boost potential BP to the potential energy surface PES in the boost-unavailable range BNPB. Moreover, the outside-barrier range BOR may be set to an interatomic distance greater than a predetermined position PP away from the pre-reaction position BRD. The predetermined position PP can be set, for example, by an empirical value based on the target atom. Furthermore, the setting unit 311 may bring the time-dependent boost potential closer to the boost potential BP illustrated in FIG. 7 by adding a plurality of boost potentials PBP illustrated in FIG. 8.

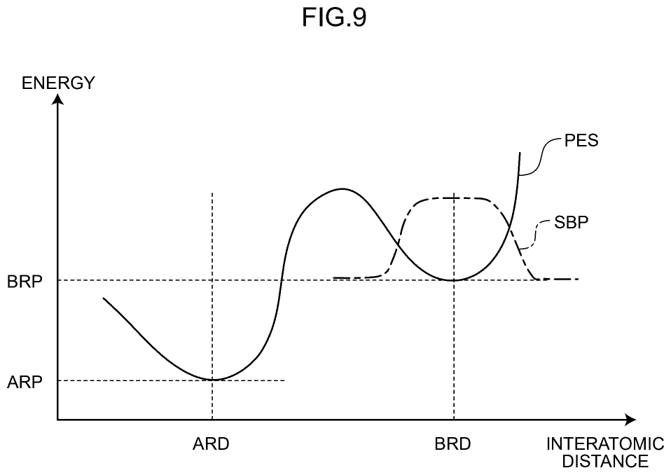

FIG. 9 is a diagram illustrating an example of a potential energy surface for an interatomic distance and an accumulated boost potential SBP in the case where the time-dependent boost potential is set as the acceleration condition. The potential energy surface PES represents the distribution of potential energy for the reaction coordinate or the collective variable. As illustrated in FIG. 9, the potential energy surface PES corresponding to the interatomic distance ARD after the chemical reaction corresponds to the potential energy ARP after the chemical reaction and reaches a minimum value. On the other hand, as illustrated in FIG. 9, the potential energy surface PES corresponding to the interatomic distance BRD before the chemical reaction corresponds to the potential energy BRP before the chemical reaction and reaches a minimum value. As illustrated in FIG. 9, the accumulated boost potential SBP is represented in an approximately trapezoidal shape by the superposition of a plurality of Gaussian functions corresponding to the multiple boost potentials.

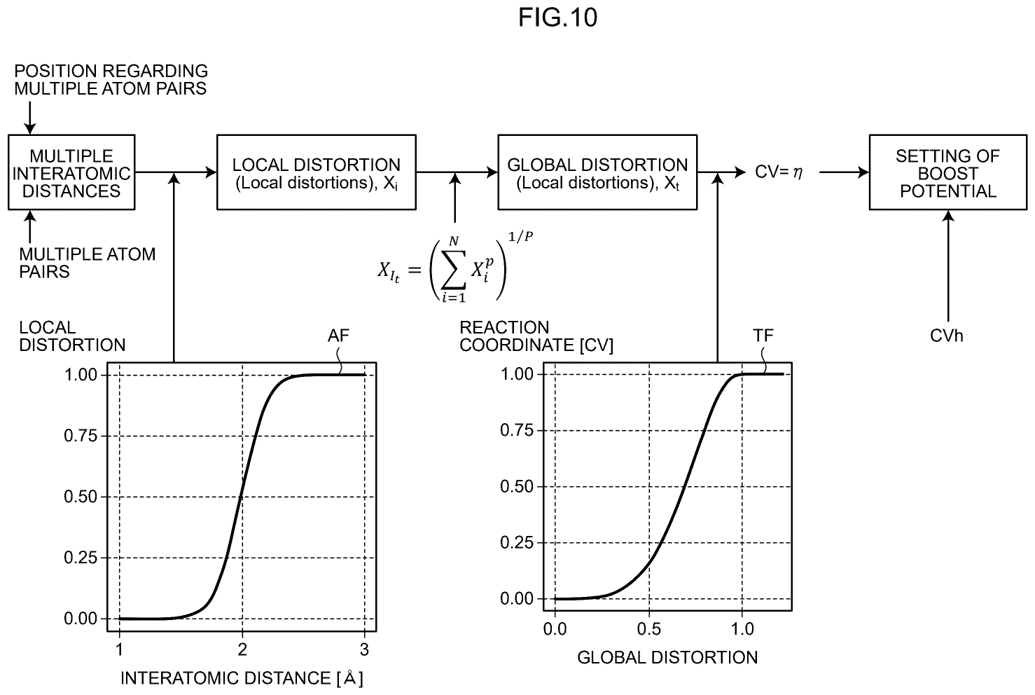

Further, in the case where the boost potential applied to the atomic group configured by a plurality of bondable atoms is a time-dependent boost potential, the setting unit 311 sets, as the acceleration condition, the shape of the activation function applied to the plurality of bondable atoms, the degree of weighting for each of multiple collective variables corresponding to the plurality of bondable atoms, or the like. An example of the acceleration condition for a case in which the plurality of bondable atoms can bond to a single reference atom (multiple atomic pairs) is now described with reference to FIG. 10.

FIG. 10 is a diagram illustrating an example of the acceleration condition that is set for the plurality of atomic pairs regarding a single reference atom. In the acceleration condition, the interatomic distance between the reference atom and each of the multiple bondable atoms is calculated based on the plurality of atomic pairs and the position regarding the plurality of atomic pairs. An activation function AF is a function that converts each of the multiple interatomic distances corresponding to the multiple bondable atoms into a local distortion Xᵢ. The subscript i in the local distortion Xᵢ is a natural number of two or more that defines the atomic pair.

The activation function AF illustrated in FIG. 10 is a function that converts the interatomic distance into 0 (zero) in the case where the reference atom and each of the multiple bondable atoms are not reacting, and into 1 (one) in the case where any of the bondable atoms is bonded to the reference atom. In other words, the local distortion Xᵢ corresponds to an index that indicates the degree of the presence or absence of a chemical bonding between each of the multiple bondable atoms and the reference atom, with a value ranging from 0 to 1, depending on the interatomic distance. Moreover, the activation function AF is not limited to being expressed by a logistic function as illustrated in FIG. 10, but may be represented using a trigonometric function.

In FIG. 10, a global distortion Xₜ is calculated by the following Formula (2) using the plurality of local distortions Xᵢ, as illustrated in FIG. 10.

$${X}_{t} = {( {\sum }_{i = 1}^{N} {X}_{i}^{p} )}^{1 / p} \qquad ( 2 )$$

As illustrated in FIG. 10, the global distortion Xₜ calculated by Formula (2) corresponds to the power mean of the local distortion Xᵢ. The power mean corresponds to an index indicating whether or not any one of the i bondable atoms is reacting. The global distortion Xₜ is a value of zero (0) if none of the bondable atoms is reacting with the reference atom, and is a value of one (1) if a single bondable atom is reacting with the reference atom.

Subsequently, as illustrated in FIG. 10, the global distortion Xₜ is converted into a reaction coordinate (also referred to as a collective variable) CV (=η) by a conversion function TF. Through this conversion, the global distortion Xₜ is converted into an index indicating whether the entire system regarding the reference atom to which the boost potential is applied is close to or far from a state in which the entire system is reacting. The setting unit 311 causes the converted reaction coordinate n to be stored in the main storage device 33 and/or the auxiliary storage device 35 along with a current time t.

As illustrated in FIG. 10, the boost potential ΔV(η) in the reaction coordinate η is calculated by the following Formula (3) using the reaction coordinate η(t) at the current time t and the reaction coordinate η(t′) at the time t′, which is minute time before the current time t. The reaction coordinate n (t′) corresponds to the history CVh of the reaction coordinate.

$$Δ V ( η ) = {\sum }_{{t}^{′} = {τ}_{G} , 2 {τ}_{G} , … , {t}^{′}} w exp [ - \frac{{( η ( t ) - η ( {t}^{′} ) )}^{2}}{2 {δ}^{2}} ] \qquad ( 3 )$$

In Formula (3), 2δ² corresponds to the range affected by the boost potential and corresponds to the half-width (full width at half maximum or half width at half maximum) of the Gaussian function. Additionally, w in Formula (3) represents the degree of weighting for each of the multiple collective variables n. Furthermore, the index indicating the range of the summation in the summation symbol Σ (t=τG, τG, 2τG, . . . , t′<t) represents the time when the MD simulation for the reference atom is executed, which is earlier than the current time t. Herein, τG represents minute time in the execution of the MD simulation. The setting unit 311 sets the range 2δ² affected by the boost potential and the weight w for each of the multiple collective variables η.

Prior to the calculation based on Formula (3), the setting unit 311 reads out the reaction coordinate η(t′), which is stored in the main storage device 33 and/or the auxiliary storage device 35, from the main storage device 33 and/or the auxiliary storage device 35. Subsequently, based on the foregoing, the setting unit 311 executes the calculation of the boost potential ΔV(η) at the reaction coordinate η in Formula (3) using the reaction coordinate η(t) at the current time t calculated by the procedure illustrated in FIG. 10, the reaction coordinate η(t′) from the past time before the current time t, the range 2δ² affected by the boost potential, and the weight w. As a result, the setting unit 311 sets the boost potential ΔV(η) applied to the plurality of atomic pairs corresponding to the identified reference atom, for example, by accumulating (summing up) the applied boost potential ΔV(η) during each execution of the MD simulation. The setting unit 311 causes the set fixed boost potential and the position to which the fixed boost potential is applied (reaction coordinate n) to be stored in the main storage device 33 and/or the auxiliary storage device 35.

### Step S307

The additional force decision unit 317 decides an additional force based on the set boost potential and the coordinates of the plurality of target atoms. Specifically, the additional force decision unit 317 decides the additional force by differentiating the boost potential at the position of the bondable electron. For example, in the case where the boost potential is set in the shape as illustrated in FIG. 6, the additional force decision unit 317 decides the action force using an analytical approach for the boost potential.

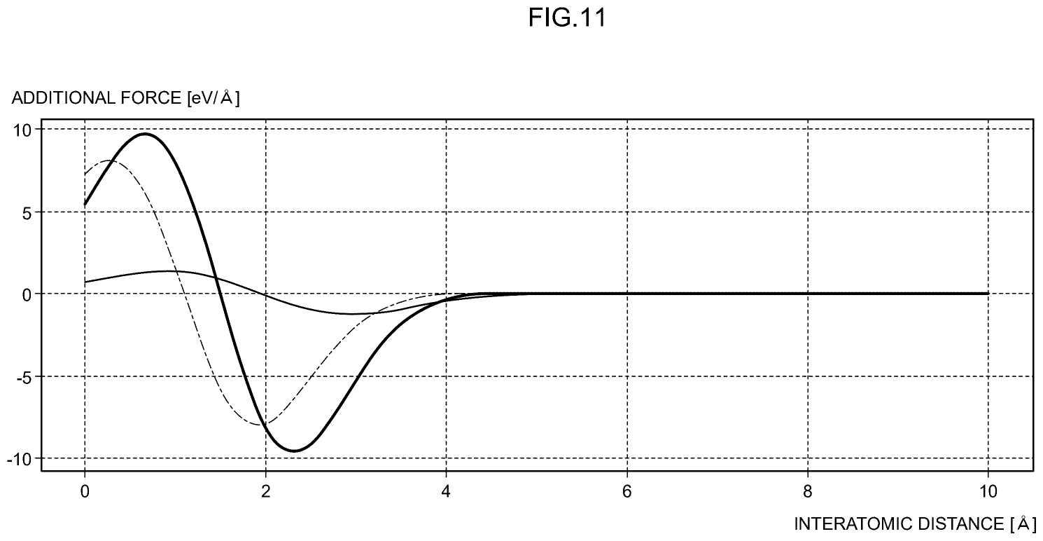

FIG. 11 is a diagram illustrating an example of the additional force corresponding to the different fixed boost potentials illustrated in FIG. 6. In FIG. 11, three additional forces respectively corresponding to the three fixed boost potentials are illustrated, each analytically calculated from the three fixed boost potentials in FIG. 6. As illustrated in FIG. 11, the additional force differs depending on the shape of the boost potential.

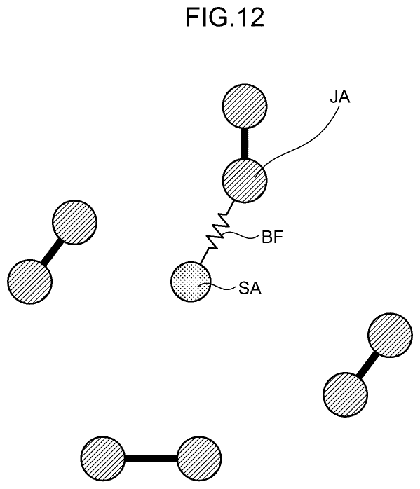

FIG. 12 is a schematic diagram illustrating an overview of the additional force in FIG. 5. As illustrated in FIG. 12, an additional force BF is set between the identified reference atom SA and the bondable atom JA. The additional force BF acts, for example, as an attractive force between the reference atom SA and the bondable atom JA. This accelerates the reaction (bond formation) between the reference atom SA and the bondable atom JA in the MD simulation described later.

### Step S308

The MD simulation unit 319 calculates the sum of the action force and the additional force. The MD simulation unit 319 uses the calculated sum of the action force and the additional force, along with the positions of the multiple atoms, to execute the MD simulation for the multiple atoms over minute time. In this case, the MD simulation unit 319 may further use a potential barrier that prevents the plurality of target atoms from separating from each other to execute the MD simulation. The execution of the MD simulation causes each of the multiple atoms to move.

### Step S309

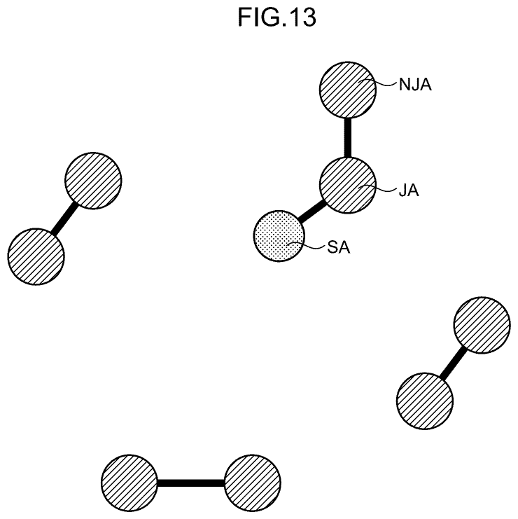

The determination unit 321 determines whether or not a chemical bonding is formed in the plurality of target atoms. For example, the determination unit 321 determines whether or not a chemical reaction is formed for at least one pair (a reference atom and one atom that can chemically bond with the reference atom). Specifically, the determination unit 321 compares the interatomic distance between the reference atom and the bond determination distance with the bond determination distance, and if a chemical bonding between the reference atom and the bondable atom is not formed (No in step S309), the processing of step S310 is executed. If a chemical bonding between the reference atom and the bondable atom is formed (Yes in step S309), the processing of step S311 is executed. FIG. 13 is a diagram illustrating an example in which a chemical bonding between the reference atom SA and the bondable atom JA is formed. As illustrated in FIG. 13, if a chemical bonding is formed, a molecule including the reference atom SA is generated. Moreover, the determination of whether or not a chemical reaction is formed is not limited to the above example, and the angles of multiple atoms (at least one pair of atoms), dihedral angles, root-mean-square deviation (RMSD) relative to a template prepared in advance, or the like may be used for the decision.

### Step S310

The determination unit 321 determines whether or not the MD simulation in step S308 is executed over a predetermined time. For example, the determination unit 321 compares the total execution time of the MD simulation with the predetermined time. If the total execution time of the MD simulation is less than the predetermined time, the processing from step S305 onwards is repeated. In this case, if a fixed boost potential is set, the processing of step S306 is omitted. On the other hand, if a time-dependent boost potential is set, in step S306, the boost potential is set again using the position of the bondable electron immediately before the execution of the MD simulation. If the sum of the execution times of the MD simulation is equal to or greater than the predetermined time, the processing of step S311 is executed. Moreover, the determination in this step is not limited to the above example, and the determination may be performed, for example, by determining whether or not the average value and/or the maximum value of the boost potential BP exceeds a value specified in advance.

Moreover, the determination unit 321 may reset the measurement of the “execution time” in step S310 to zero (0) in the case where the decision in step S310 results in Yes, that is, upon the execution time reaching a predetermined time. In other words, the determination unit 321 may restart the count of the execution time during the loop processing from step S305 to step S309. Additionally, there may be two types of execution time of the total execution time in the MD simulation execution processing and the execution time used for the determination in step S310. In this case, the latter execution time may be used for the determination in step S310.

### Step S311

The determination unit 321 initializes the sum of the boost potentials applied to a plurality of target atoms. For example, the determination unit 321 initializes the sum of the fixed boost potential and the time-dependent boost potential added during each execution of the MD simulation.

### Step S312

The identification unit 313 updates the attribute information of the atom with which the chemical bonding is formed, based on the functional group regarding the atoms with which the chemical bonding is formed. In this way, the identification unit 313 updates the plurality of target atoms. For example, in FIG. 13, if a chemical bonding is determined to be formed between the reference atom SA and the bondable atom JA, the identification unit 313 updates the attribute information of the bondable atom using a program based on a predetermined rule for the chemically bonded functional group, thereby identifying a new bondable atom NJA. After this step, the processing from step S304 onwards is repeated. In other words, in the case where the determination unit 321 determines that a chemical bonding is formed, the identification unit 313 updates the attribute information of the plurality of target atoms with which the chemical bonding is formed, and then identifies the other multiple target atoms.

### Step S313

The determination unit 321 determines whether or not the specified atomic attribute (i.e., the updated attribute) is equal to or less than a certain number. Specifically, if the total number of the multiple reference atoms (e.g., atomic pairs) identified by the identification unit 313 is 100 and the certain number is 10, the determination unit 321 determines whether or not a boost potential is set for each of the 90 reference atoms. If the specified atomic attribute (i.e., the updated attribute) is not equal to or less than the certain number (No in step S313), the processing from Step S304 onwards is executed. In this case, if a chemical bonding is determined to be formed, the identification unit 313 identifies other multiple target atoms that are subject to chemical bonding among the multiple atoms (step S304). Subsequently, the action force decision unit 315 inputs the atomic structures of the multiple atoms after the MD simulation into the trained NNP to decide the second action force acting on each of the multiple atoms (step S305). In addition, the additional force decision unit 317 decides information regarding the second additional force to be applied to at least one of the other target atoms (steps 306 and S307). Subsequently, the MD simulation unit 319 executes a molecular dynamics simulation for the multiple atoms again using the second additional force, the information regarding the second additional force, and the position information of the multiple atoms after the execution of the MD simulation.

In other words, the processing of steps S304 through S312, such as, for example, identifying a plurality of target atoms, acquiring information regarding an action force, acquiring information regarding an additional force, and executing a molecular dynamics simulation, is repeatedly executed until a predetermined criterion is satisfied. If the specified atomic attribute (i.e., updated attribute) exceeds a certain number (Yes in step S313), the processing of step S314 is executed.

In the above processing, there may be cases where a determination of “No” in Step S313 continues. For example, if the number of atoms involved in the reaction is extremely small, or in the case where the atoms and molecules hardly move in the simulation space (such as the case where the viscosity due to the atoms or molecules is high), the case where the determination result is “No” is liable to continue. For this reason, the maximum number of loops (a predetermined number of iterations) for the processing from step S304 to step S313 may be pre-set (specified). In this case, in the determination of step S313, if the number of loops of the processing from step S304 to step S313 reaches the predetermined number of times, or is performed equal to or more than the predetermined number of times, the processing of step S314 is executed.

### Step S314

The evaluation unit 323 evaluates the physical property (physical characteristic) of the molecule generated by the chemical reaction accompanying the MD simulation. The evaluation unit 323 outputs the evaluation of the physical properties of the molecule to the main storage device 33, the auxiliary storage device 35, and/or a display. Moreover, in addition to displaying the evaluation of the physical properties on the display, a moving image (animation) representing the movement of the multiple atoms during the MD simulation may be displayed on the display in chronological order.

From the above, the information processing apparatus 1 according to the present embodiment identifies a plurality of target atoms that is subject to chemical bonding among multiple atoms, acquires information regarding the first action force acting on each of the multiple atoms, which is generated by inputting the atomic structure of the multiple atoms into a trained neural network potential, acquires information regarding the first additional force acting on at least one of the plurality of target atoms, and executes an MD simulation for the multiple atoms using the information regarding the first action force, the information regarding the first additional force, and the position information of the multiple atoms. Furthermore, the information processing apparatus 1 according to the present embodiment determines whether or not a chemical bonding is formed for the plurality of target atoms based on the position information of the plurality of target atoms after the execution of the MD simulation.

As a result, the information processing apparatus 1 according to the present embodiment enables a boost potential to be applied between atoms that are desired to induce a reaction, and to execute an MD simulation using an additional force based on the boost potential and an action force based on the NNP. Accordingly, the information processing apparatus 1 according to the embodiment makes it possible for a chemical reaction to be generated in a practical calculation time for the MD simulation and for a chemical reaction such as polymerization and decomposition to be handled for a broad range of substances.

Further, the information processing apparatus 1 according to the embodiment, in the case where a chemical bonding between the target atoms is determined not to be formed, acquires information regarding the second action force acting on each of the multiple atoms, which is generated by inputting the multiple atomic structure after the execution of the MD simulation into a neural network, acquires information regarding the second additional force acting on at least one of the plurality of target atoms, and re-executes the MD simulation for the multiple atoms using the information regarding the second action force, the information regarding the second additional force, and the position information of the multiple atoms after the execution of the MD simulation. Furthermore, the information processing apparatus 1 according to the embodiment, in the case where a chemical bonding between the target atoms is determined not to be formed, executes the re-executed MD simulation by further using the information regarding the first additional force.

Thus, the information processing apparatus 1 according to the embodiment makes it possible to execute the MD simulation using the additional force decided by accumulating the boost potential as the MD simulation is executed. For this reason, the information processing apparatus 1 according to the embodiment makes it possible to further improve the acceleration (boost) of the chemical reaction, and further reduce the calculation time for generating the chemical reaction in the MD simulation.

Further, the information processing apparatus 1 according to the embodiment, in the case where a chemical bonding between the target atoms is determined not to be formed, identifies the other multiple target atoms that are subject to chemical bonding among the multiple atoms, acquires information regarding the second action force acting on each of the multiple atoms, which is generated by inputting the atomic structure of the multiple atoms after the MD simulation is performed into a trained neural network, acquires information regarding the second additional force acting on at least one of the other multiple target atoms, and re-executes the MD simulation for the multiple atoms using the information regarding the second action force, the information regarding the second additional force, and the position information of the multiple atoms after the execution of the MD simulation. As a result, the information processing apparatus 1 according to the embodiment makes it possible to efficiently apply the additional force to the bondable atom by dynamically applying the boost potential based on the movement history of the bondable atoms (metadynamics), thereby further reducing the calculation time for generating a chemical reaction in the MD simulation.

Further, the information processing apparatus 1 according to the embodiment, in the case where the distance between the target atoms is less than a predetermined threshold, decides, as the information regarding the first additional force, that the first additional force is not to be applied to the plurality of target atoms. This makes it possible for the information processing apparatus 1 according to the embodiment to, in the case where the bondable electron approaches the reference atom up to the covalent bond radius, execute the MD simulation without applying the boost potential and the additional force based on the boost potential. Thus, the information processing apparatus 1 according to the embodiment enables a chemical reaction to occur in a practical calculation time by executing the MD simulation under a naturally occurring situation.

Additionally, the information processing apparatus 1 according to the embodiment executes the MD simulation further using a potential barrier that prevents the coordinates of the target atoms from separating from each other. As a result, the information processing apparatus 1 according to the embodiment makes it possible to prevent the reference atom and the bondable atom from separating more than necessary by applying the boost potential, thereby enabling a chemical reaction to occur in a practical calculation time.

Further, the information processing apparatus 1 according to the embodiment decides information regarding the first additional force based on the position information of the plurality of target atoms. Specifically, the information regarding the first additional force is decided based on the position information of the plurality of target atoms and the boost potential. In other words, the information processing apparatus 1 according to the embodiment makes it possible to apply a boost potential to the plurality of bondable atoms with respect to the reference atom. As a result, the information processing apparatus 1 according to the embodiment enables the occurrence of a chemical reaction more efficiently in a practical calculation time.

Further, the information processing apparatus 1 according to the embodiment executes the MD simulation for the multiple atoms using the sum of the first action force and the first additional force as well as the position information of the multiple atoms. For example, the information processing apparatus 1 according to the embodiment, in the case where a chemical bonding is determined to be formed, initializes the boost potential, identifies other target atoms that are subject to chemical bonding among the multiple atoms, decides the second action force acting on each of the multiple atoms by inputting the types of the multiple atoms and the positions of the multiple atoms into the trained neural network potential, decides the second additional force acting on each of the other multiple target atoms based on the coordinates of the other multiple target atoms, executes a molecular dynamics simulation for the multiple atoms using the sum of the second action force and the second additional force and the positions of the multiple atoms, and determines whether or not a chemical bonding is formed based on the positions of the plurality of target atoms after the execution of the MD simulation.

Thus, the information processing apparatus 1 according to the embodiment is capable of suppressing the reverse reaction (decomposition reaction) of the chemical bonding by initializing the boost potential upon each occurrence of a chemical bonding formation. Accordingly, the information processing apparatus 1 according to the embodiment makes it possible to apply the influence of the application of the boost potential efficiently to the other multiple target atoms, thereby enabling a chemical reaction to occur in a more practical calculation time.

### Application Example

The present application example involves identifying a plurality of target atoms by using a structure up to a middle layer at the final stage (hereinafter referred to as a final middle layer) of a trained neural network potential (first neural network) and by using a trained neural network (hereinafter referred to as a specific neural network (second neural network)) using an output layer connected to the stage following the final middle layer. The input to the specific neural network is the position of multiple atoms arranged in a virtual space and the type (atomic structure) of the multiple atoms, as in the NNP. Moreover, the middle layer connected to the output layer in the specific neural network is not limited to the final middle layer, and may be another middle layer. The specific neural network is generated by learning (e.g., transfer learning) using the feature values of the middle layer calculated by the NNP. The output from the specific neural network (output information) is, for example, a label (label information) indicating the type of functional group (type of atom) that includes multiple atoms that are more likely to form a chemical bonding (chemical reaction). Examples of such labels include “carbon radical”, “carbon of vinyl group”, or the like.

The identification unit 313 receives, as input to the specific neural network, the position of multiple atoms arranged in the virtual space and the type of the multiple atoms, and outputs a plurality of labels corresponding to the multiple atoms. In the case where a label regarding a chemical bonding (functional group) is specified by a user instruction via the input interface, the identification unit 313 identifies the label corresponding to the specified functional group from among the multiple labels output by the specific neural network. The label corresponds to the identifier of the target atom. Subsequently, the identification unit 313 identifies a plurality of target atoms using the search range, with the atom corresponding to the identified label serving as the reference atom. As a result, the identification unit 313 identifies the plurality of target atoms using the atomic structure of the multiple atoms and the trained second neural network. In other words, the identification unit 313 identifies the plurality of target atoms based on output information from the trained second neural network. In this case, the identification unit 313 acquires the output information by inputting the atomic structure of the multiple atoms into the trained second neural network. Since the identification of the plurality of target atoms using the search range is similar to the embodiment, further description is omitted.

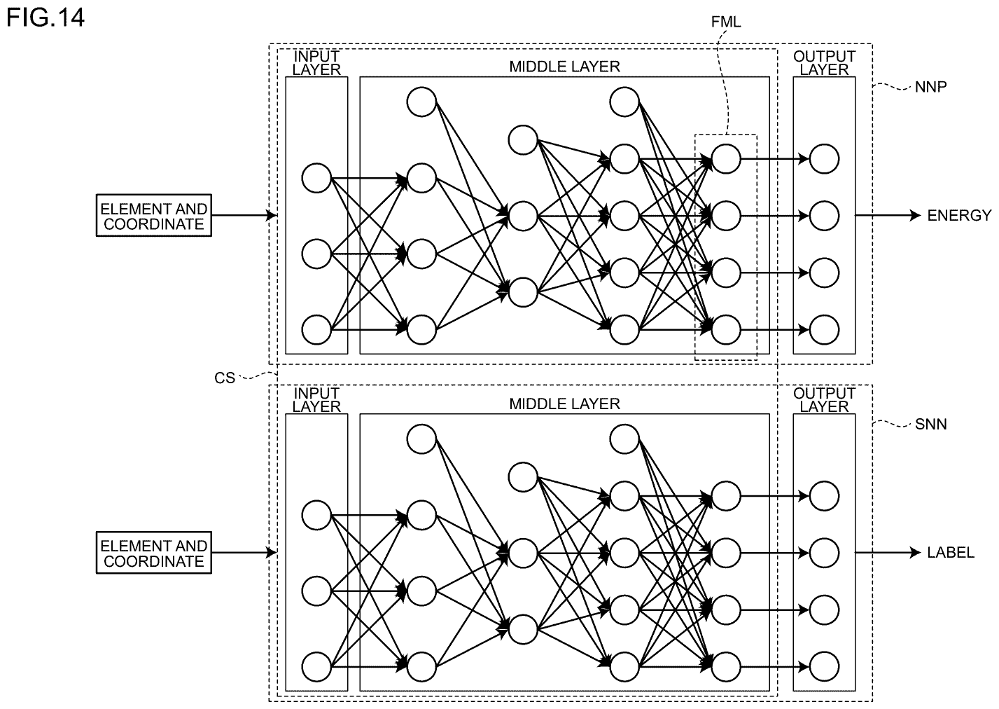

FIG. 14 is a diagram illustrating an example of an NNP (first neural network) and a specific neural network (second neural network) SNN. As illustrated in FIG. 14, the NNP receives, as input, the position of multiple atoms and the type of the multiple atoms, and outputs the energy of each atom. On the other hand, as illustrated in FIG. 14, the input layer and the middle layer are shared between the NNP and the specific neural network SNN. The difference between the specific neural network SNN and the NNP is that the output destination from a final middle layer FML of the NNP is an output layer distinct from that of the NNP. As illustrated in FIG. 14, in the specific neural network SNN, a label is generated using the middle layer of the NNP in which the local positional relationships between respective atoms are output as feature values and using the input of the NNP. Thus, the feature value obtained by the NNP may also potentially include information regarding a functional group. Moreover, the feature value of the NNP is generated, for example, by TeaNet or a reaction neural network.

The specific neural network SNN is generated by learning, which uses the position of multiple atoms and the type of the multiple atoms as training data and uses data indicating the preset label corresponding to the training data as correct answer data. This learning conforms to, for example, known transfer learning, so further description is omitted.

Moreover, as a modification of the application example, the identification unit 313 may acquire output information by inputting, into the trained neural network (another neural network), information (feature values) obtained from the middle layer of the trained neural network (first neural network) upon inputting the atomic structure of multiple atoms into the trained neural network (first neural network). The other neural network and the specific neural network SNN may be collectively referred to as the second neural network. In this case, if the input information to the second neural network is an atomic structure, the second neural network corresponds to the specific neural network SNN. Furthermore, in the case where the input information to the second neural network is information from the middle layer of the NNP (first neural network), the second neural network corresponds to the other neural network. Additionally, while the above description describes the output information being output from the second neural network as label information for each input atom, the output information is not limited to this example. The output information being output from the second neural network may be output as information regarding whether or not a reaction is possible or not for each of the input atoms (multiple atoms being input into the input layer of the first neural network).

From the above, the information processing apparatus 1 according to the application example of the present embodiment identifies a plurality of target atoms using the atomic structure of the multiple atoms and the trained second neural network. For example, the information processing apparatus 1 according to the application example of the present embodiment identifies a plurality of target atoms based on output information from the trained second neural network. More specifically, the information processing apparatus 1 according to the application example of the present embodiment acquires output information by inputting the atomic structure of the multiple atoms into the trained second neural network. Moreover, the information processing apparatus 1 according to the application of the present embodiment may acquire the output information by inputting, into the trained second neural network, information obtained from the middle layer of the trained neural network upon inputting the atomic structures of the multiple atoms into the trained neural network. The information processing apparatus 1 according to the application of the present embodiment makes it possible to identify a plurality of target atoms automatically by the user's selection of the type of functional group.

As a result, the information processing apparatus 1 according to the application example of the present embodiment eliminates the need for the user to specify a plurality of reference atoms in the equilibrated initial structure INS placed within the simulation space SS, as illustrated in FIG. 4, without the need for substructure matching using techniques such as SMARTS, thereby reducing the preparation burden on the user before execution of the MD simulation. Other effects are similar to those described in the embodiment, and thus further description is omitted.

In the case where the technical concept according to the embodiment is implemented by an information processing method, the information processing method that causes at least one computer to perform: identifying a plurality of target atoms subject to chemical bonding among the multiple atoms; acquiring information regarding a first action force acting on each of the multiple atoms, the information being generated by inputting an atomic structure of the multiple atoms into a trained neural network potential; acquiring information regarding a first additional force acting on at least one of the plurality of target atoms; and executing a molecular dynamics simulation for the multiple atoms using the information regarding the first action force, the information regarding the first additional force, and position information of the multiple atoms. The procedure and effects of the MD simulation execution processing regarding the information processing method are similar to those described in the embodiment, and thus further description is omitted.

In the case where the technical concept according to the embodiment is implemented by an information processing program, the information processing program causes at least one computer to perform: identifying a plurality of target atoms subject to chemical bonding among the multiple atoms; acquiring information regarding a first action force acting on each of the multiple atoms, the information being generated by inputting an atomic structure of the multiple atoms into a trained neural network potential; acquiring information regarding a first additional force acting on at least one of the plurality of target atoms; and executing a molecular dynamics simulation for the multiple atoms using the information regarding the first action force, the information regarding the first additional force, and position information of the multiple atoms.

For example, it is possible to implement the information processing program by installing the information processing program in a computer of a simulation device, a simulation server, or the like that executes an MD simulation for multiple atoms and by loading the program into memory. In this case, the program capable of causing a computer to execute the MD simulation execution processing can also be stored and distributed on a storage medium such as a magnetic disk (such as hard disk), an optical disk (such as CD-ROM or DVD), or a semiconductor memory. The procedure and effects of the MD simulation execution processing using the information processing program are the same as those described in the embodiment, and thus further description is omitted.

The respective components or devices in the embodiments described above, in whole or in part, may be configured with hardware, or may be configured with information processing via software (programs) executed by a CPU, GPU, or the like. In the case where information processing is configured by software, the software that implements at least some of the functions of each component or device in the above-mentioned embodiment may be stored on a non-transitory computer-readable medium such as a flexible disk, compact disc-read only memory (CD-ROM), or USB memory, and loaded into the computer 30 to execute the information processing configured by the software. Alternatively, the software may also be downloaded via the communication network 5. Furthermore, the software may be implemented in a circuit such as an ASIC or FPGA, enabling the hardware-based execution of information processing.

The type of storage medium that stores the software is not limited to a particular format. The storage medium is not limited to removable media such as a magnetic disk or optical disk, but may be a fixed storage medium such as a hard disk or memory. Additionally, the storage medium may be provided internally within the computer or may be provided externally to the computer.

In the present specification (including the claims), in the case where the expressions “at least one of a, b, and c” or “at least one of a, b, or c” (or similar expressions) are used, they are construed to encompass any of a, b, or c alone; any combination of a-b, a-c, and b-c; or a combination of a-b-c. Additionally, the expressions may encompass multiple instances of any of the elements or components, such as a, a; a, b, b; or a, a, b, b, c, c (a-a, a-b-b, a-a-b-b-c-c). Furthermore, the expressions encompass an additional element or component beyond the enumerated elements or components (a, b, and c), such as the inclusion of d in a, b, c, and d (a-b-c-d).

In the present specification (including the claims), in the case where the expressions such as “using data as input”, “based on data”, “depending on data”, or “in response to data” (or similar expressions) are used, they encompass, unless otherwise specified, the case where various types of data themselves are used as input and the case where data obtained by performing certain processing on such data (e.g., such as data with noise added, normalized data, or intermediate representations of various types of data) are used as input. In addition, in the case where a certain result is described as being obtained “based on data”, “depending on data”, or “in response to data”, this is construed to encompass the case where the result is obtained solely based on the data as well as the case where the result is obtained under the influence of other data, factors, conditions, and/or states beyond the data. Furthermore, in the case where “data is output” is described, this is construed, unless otherwise stated, to encompass the case where the various types of data themselves are used as output and the case where data obtained by performing certain processing on such data (e.g., such as data with noise added, normalized data, or intermediate representations of various types of data) are used as output.

In the present specification (including the claims), in the case where the terms “connected” and “coupled” are used, the terms are intended to be non-limiting terms that encompass any direct connection or coupling, indirect connection or coupling, electrical connection or coupling, communicative connection or coupling, operative connection or coupling, physical connection or coupling, and the like. These terms should be construed as appropriate depending on the context in which they are used, and any forms of connection or coupling that are not intentionally or inherently excluded should be non-limitedly construed as being included in the scope of meaning of these terms.

In the present specification (including the claims), in the case where the expression “A configured to B” is used, this expression may encompass that the physical structure of component A has a configuration capable of executing operation B, as well as may encompass that the permanent or temporary setting or configuration of component A is configured or set to actually execute operation B. For example, in the case where component A is a general-purpose processor, it is sufficient that the processor has a hardware configuration capable of executing operation B and is configured to actually execute operation B by the setting of a permanent or temporary program (instruction). Furthermore, in the case where component A is a dedicated processor, dedicated computational operation circuit, or the like, it is sufficient that the circuit structure of the processor is implemented or established to actually execute operation B, regardless of whether or not control instructions and data are actually attached.

In the present specification (including the claims), in the case where the terms indicating inclusion or possession (such as “comprising”, “including” and “having”) are used, such terms are intended to be open-ended terms that encompass the case where an object or target other than that indicated by the object of such terms is included or possessed. In the case where the object of such terms meaning inclusion or possession is an expression that does not specify a quantity or suggests singularity (an expression with “a” or “an” as an article), the expression should be construed as not being limited to a specific number.

In the present specification (including the claims), even if an expression such as “one or more” or “at least one” is used in one portion of the specification and the expression that does not specify a quantity or suggest singularity (expressions with “a” or “an” as an article) is used in another portion of the specification, the latter expression is not intended to mean “one”. In general, expressions that do not specify a quantity or suggest singularity (expressions with “a” or “an” as an article) should be construed as not necessarily being limited to a specific number.

In the present specification, in the case where a specific effect (advantage or result) is described as being obtained from a particular configuration in a given embodiment, it should be understood that, unless otherwise indicated, the same effect is also obtained in one or more other embodiments that include the same configuration. However, the presence or absence of such an effect generally depends on various factors, conditions, and/or states, and it should be understood that the same effect is not necessarily obtained solely by the same configuration. The effect is merely obtained from the configuration described in the embodiments when various factors, conditions, and/or states are satisfied, and it should not be assumed that the effect is necessarily obtained in the invention pertaining to the claims defining such a configuration or a similar configuration.

In the present specification (including the claims), in the case where the terms such as “maximize” are used, they encompass obtaining a global maximum value, obtaining an approximation of a global maximum value, obtaining a local maximum value, and obtaining an approximation of a local maximum value, and should be construed as appropriate depending on the context in which the terms are used. They also encompass obtaining approximate values of the maximum values through probabilistic or heuristic methods. Similarly, in the case where the terms like “minimize” are used, they encompass obtaining a global minimum value, obtaining an approximation of a global minimum value, obtaining a local minimum value, and obtaining an approximation of a local minimum value, and should be construed as appropriate depending on the context in which the terms are used. They also encompass obtaining approximate values of the minimum values through probabilistic or heuristic methods. Similarly, in the case where terms such as “optimize” are used, they encompass obtaining a global optimum value, obtaining an approximation of a global optimum value, obtaining a local optimum value, and obtaining an approximation of a local optimum value, and should be construed as appropriate depending on the context in which the terms are used. They also encompass obtaining approximate values of the optimum values through probabilistic or heuristic methods.

In the present specification (including claims), in the case where multiple hardware devices perform predetermined processing, the hardware devices may cooperate with each other to perform the predetermined processing, or a portion of the hardware devices may perform all of the predetermined processing. Additionally, some hardware devices may perform a portion of the predetermined processing, and another portion of the hardware devices may perform the remainder of the predetermined processing. In the present specification (including claims), in the case where expressions such as “one or more hardware devices perform first processing, and the one or more hardware devices perform second processing” are used, the hardware device performing the first processing and the hardware device performing the second processing may be the same or different. In other words, it is sufficient that the hardware device performing the first processing and the hardware device performing the second processing are included in one or more hardware devices. Moreover, the hardware device may include an electronic circuit or a device incorporating electronic circuits.

In the present specification (including the claims), in the case where multiple storage devices (memories) store data, each storage device (memory) of the multiple storage devices (memories) may store only a portion or the entirety of the data.

Although the invention has been described with respect to specific embodiments for a complete and clear disclosure, the appended claims are not to be thus limited but are to be construed as embodying all modifications and alternative constructions that may occur to one skilled in the art that fairly fall within the basic teaching herein set forth.

What is claimed is:

1. An information processing apparatus comprising:
at least one memory; and
at least one processor, wherein
the at least one processor is configured to:
identify a plurality of target atoms subject to chemical bonding among a plurality of atoms;
acquire information regarding a first action force acting on each of the plurality of atoms, the information being generated by inputting an atomic structure of the plurality of atoms into a neural network;
acquire information regarding a first additional force to be applied to at least one of the plurality of target atoms; and
execute a molecular dynamics simulation for the plurality of atoms using the information regarding the first action force, the information regarding the first additional force, and position information of the plurality of atoms.

2. The information processing apparatus according to claim 1, wherein
the at least one processor is configured to
determine whether or not the chemical bonding of the plurality of target atoms is formed based on position information of the plurality of target atoms after execution of the molecular dynamics simulation.

3. The information processing apparatus according to claim 2, wherein
the at least one processor is configured to: in a case where the chemical bonding of the plurality of target atoms is determined not to be formed,
acquire information regarding a second action force acting on each of the plurality of atoms, the information being generated by inputting an atomic structure of the plurality of atoms after execution of the molecular dynamics simulation into the neural network;
acquire information regarding a second additional force to be applied to at least one of the plurality of target atoms; and
re-execute the molecular dynamics simulation for the plurality of atoms using the information regarding the second action force, the information regarding the second additional force, and position information of the plurality of atoms after execution of the molecular dynamics simulation.

4. The information processing apparatus according to claim 3, wherein
the at least one processor is configured to, in the case where the chemical bonding of the plurality of target atoms is determined not to be formed, re-execute the molecular dynamics simulation by further using the information regarding the first additional force.

5. The information processing apparatus according to claim 1, wherein
the at least one processor is configured to execute the molecular dynamics simulation by further using a potential barrier that prevents the plurality of target atoms from separating from each other.

6. The information processing apparatus according to claim 1, wherein
the at least one processor is configured to: in a case where the chemical bonding is determined to be formed,
identify a plurality of other target atoms subject to chemical bonding among the plurality of atoms;
acquire information regarding a second action force acting on each of the plurality of atoms, the information being generated by inputting, into the neural network, an atomic structure of the plurality of atoms after execution of the molecular dynamics simulation;
acquire information regarding a second additional force to be applied to at least one of the plurality of other target atoms; and
re-execute the molecular dynamics simulation for the plurality of atoms using the information regarding the second action force, the information regarding the second additional force, and position information of the plurality of atoms after execution of the molecular dynamics simulation.

7. The information processing apparatus according to claim 6, wherein
the at least one processor is configured to, in the case where the chemical bonding is determined to be formed, update attribute information of the plurality of target atoms with which the chemical bonding is formed and then identify the plurality of other target atoms.

8. The information processing apparatus according to claim 1, wherein
the at least one processor is configured to, in a case where a distance between the plurality of target atoms is less than a predetermined threshold, decide, as the information regarding the first additional force, that the first additional force is not to be applied to the plurality of target atoms.

9. The information processing apparatus according to claim 1, wherein
the at least one processor is configured to repeatedly execute, until a predetermined criterion is satisfied,
identification of a plurality of target atoms, acquisition of information regarding an action force, acquisition of information regarding an additional force, and execution of a molecular dynamics simulation.

10. The information processing apparatus according to claim 1, wherein
the at least one processor is configured to decide the information regarding the first additional force based on the position information of the plurality of target atoms.

11. The information processing apparatus according to claim 1, wherein
the at least one processor is configured to decide the information regarding the first additional force based on the position information of the plurality of target atoms and a boost potential.

12. The information processing apparatus according to claim 1, wherein
the at least one processor is configured to
execute the molecular dynamics simulation for the plurality of atoms using a sum of the first action force and the first additional force, and the position information of the plurality of atoms.

13. The information processing apparatus according to claim 1, wherein
the at least one processor is configured to
identify the plurality of target atoms using the atomic structure of the plurality of atoms and a second neural network.

14. The information processing apparatus according to claim 13, wherein
the at least one processor is configured to
identify the plurality of target atoms based on output information from the second neural network.

15. The information processing apparatus according to claim 14, wherein
the at least one processor is configured to
acquire the output information by inputting the atomic structure of the plurality of atoms into the second neural network.

16. The information processing apparatus according to claim 14, wherein
the at least one processor is configured to
acquire the output information by inputting, into the second neural network, information obtained from a middle layer of the neural network upon inputting the atomic structure of the plurality of atoms into the neural network.

17. The information processing apparatus according to claim 1, wherein
the at least one processor is configured to input the atomic structure of the plurality of atoms into the neural network and generate the information regarding the first action force acting on each of the plurality of atoms.

18. An information processing method comprising:
by at least one computer,
identifying a plurality of target atoms subject to chemical bonding among a plurality of atoms;
acquiring information regarding a first action force acting on each of the plurality of atoms, the information being generated by inputting an atomic structure of the plurality of atoms into a neural network;
acquiring information regarding a first additional force to be applied to at least one of the plurality of target atoms; and
executing a molecular dynamics simulation for the plurality of atoms using the information regarding the first action force, the information regarding the first additional force, and position information of the plurality of atoms.

19. The information processing method according to claim 18, further comprising, by the at least one computer, identifying the plurality of target atoms using the atomic structure of the plurality of atoms and a second neural network.

20. A non-transitory computer-readable medium including programmed instructions that cause at least one computer to perform:
identifying a plurality of target atoms subject to chemical bonding among a plurality of atoms;
acquiring information regarding a first action force acting on each of the plurality of atoms, the information being generated by inputting an atomic structure of the plurality of atoms into a neural network;
acquiring information regarding a first additional force to be applied to at least one of the plurality of target atoms; and
executing a molecular dynamics simulation for the plurality of atoms using the information regarding the first action force, the information regarding the first additional force, and position information of the plurality of atoms.
